(19) United States
(12) Patent Application Publication
(10) Pub. No.: US 2021/0371899 A1
(43) Pub. Date: Dec. 2, 2021

(54) METHOD AND KIT FOR PESTICIDES DETECTION, AND PLASMID, BACULOVIRUS, CELL AND METHOD OF PREPARING THE SAME FOR PESTICIDE DETECTIONS

(71) Applicant: ACADEMIA SINICA, Taipei (TW)

(72) Inventors: Yu-Chan CHAO, Taipei (TW); Lin-Li LIAO, Taipei (TW); Chuan-yu LIAO, Taipei (TW); Chih-Hsuan TSAI, Taipei (TW); Paul Wei-che HSU, Taipei (TW)

(73) Assignee: ACADEMIA SINICA, Taipei (TW)

(21) Appl. No.: 17/285,945

(22) PCT Filed: Oct. 17, 2019
(86) PCT No.: PCT/US2019/056657

§ 371 Date: Apr. 16, 2021

Related U.S. Application Data
(60) Provisional application No. 62/747,258, filed on Oct. 18, 2018.

(51) Int. Cl. C12Q 1/46; C12N 9/18; G06N 20/10; G01N 21/78

(52) U.S. Cl. CPC C12Q 1/46; G01N 21/78; G06N 20/10; C12N 9/18

(57) ABSTRACT

This disclosure provides a method and a kit for pesticide detection. By expressing acetylcholinesterases on cell surface, rapid pesticide screening, identification and quantification of pesticides or insecticides may be achieved.

### CROSS-REFERENCE TO RELATED APPLICATION

This application claims the benefit of U.S. Provisional Application No. 62/747,258, filed Oct. 18, 2018.

### FIELD OF INVENTION

This present disclosure generally relates to the field of detecting hazardous pesticides, insecticides or the like. More particularly, the present disclosure relates to a method and a kit for pesticides, insecticides or the like detection, and a plasmid, a baculovirus, a cell and a method of preparing the same for detection of pesticides, insecticides or the like.

### BACKGROUND OF THE INVENTION

Pesticides or insecticides are used commonly in agriculture to control insect pests that threatening crop production and/or carry diseases[1]. With intensive utilization of insecticides, the residues in agriculture products could damage human and other animals' health which lead to significant ecotoxicological problems. Organophosphorus (OP) and carbamate (CB) insecticides are the most commonly used pesticides due to their high effectiveness and low persistence[2]. OP and CB insecticides have been developed to block the active sites of the acetylcholinesterase (AChE; EC 3.1.1.7) in insects. AChE is a membrane associated enzyme that terminates nerve impulses by catalyzing the hydrolysis of the neurotransmitter acetylcholine in the synaptic cleft. OP and CB insecticides phosphorylate or carbamoylate the serine of the catalytical triad, and thus preventing the termination of a nerve impulse in the postsynaptic membrane, which results in acetylcholine accumulation and continuous stimulation of the insect nervous system eventually leading to the death of insects[3]. OP and CB insecticides are therefore also toxic to human beings because the vertebrate AChEs are similar in structure and function to the insect enzymes[4]. Structures of the main pesticides which blocks the active sites of the AChEs are shown below.

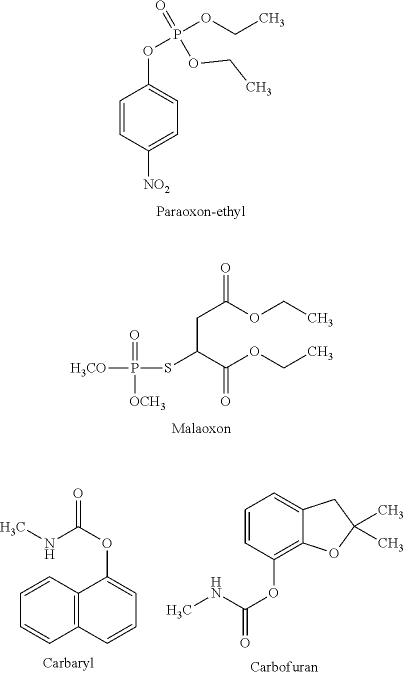

Since AChE is the target of OP and CB compounds, detection of insecticides residue could therefore depends on the hydrolysis ability of AChE. Neurotransmitter acetylcholine will be catalytically hydrolyzed by AChE to thiocholine. By applying the Ellman's reagent DTNB (5,5′-dithio-bis-[2-nitrobenzoic acid]) to estimate sulfhydryl groups in the solution, a detectable yellow-colored product TNB (2-nitro-5-thiobenzoic acid) will yield which could be measured at OD 412 nm[5]. Upon the presence of OP or CB insecticides, the hydrolysis process of acetylcholine by AChE will be blocked and the formation of yellow-colored product will be inhibited.

To strictly regulate and prevent accidental exposure of people and livestock to these toxicants, inspection institutes require a sufficient supply of AChE for examining whether agricultural products contain any residue pesticides exceeding allowance. Currently, the most common source of AChE is extracted from the head of house-fly, which requires fly breeding, extraction, sucrose gradient centrifugation, enzyme purification and activity assay[6]. This is an expensive, laborious, and time-consuming process. In addition, AChE yields could be low due to lengthy and tedious purification procedures. Moreover, purified AChEs must be made into powder or be coated on the wells of plates for preservation, shipping and application. AChE powder has to be solubilized and applied into wells of reaction plates before detection of residue insecticide. Therefore, a better strategy for the detection of insecticide residues is needed.

### SUMMARY OF THE INVENTION

Baculovirus is a versatile tool for agricultural and biotechnological applications. It has long been served as a microorganism for insect pest control in the fields. Baculovirus is also served as one of the major tools for the production of the engineered proteins[7]. This system can produce proteins with high yield and proper post-translational modifications that are suitable for various applications such as vaccine, experimental protein, industrial protein, reagent for detection kits, etc.[8]. Autographa californica multiple nucleopolyhedrovirus (AcMNPV) is the type baculovirus species which infects only lepidopteran insects and cell lines. This virus has a double-stranded, closed-circular DNA genome of 134 kb with a coding capacity of over 154 polypeptides[9].

In view of the needs in the art, the inventors aimed to develop a novel cell-based detection system which is highly-sensitive for examination of insecticides residue, e.g. OP and CB, by displaying AChE on the surface of cells, for example insect cells, using recombinant baculoviruses. Since AChE is displayed on cell surface for functional detection of insecticides, AChE extraction and purification are not needed, the production time and cost for the entire detection system could be reduced. The conditions of recombinant virus infection and effects of lyophilization were analyzed to optimize the detection system.

In the present application, the inventors develop a method and a kit for detection of pesticide, e.g. organophosphates (OP) and carbamates (CB), two of the major insecticides frequently applied in agriculture practice. The present application provides a convenient and rapid solution for the detection of pesticide including insecticides. In addition, the inventors further develop a method and pesticide rapid screening system that can determine both the pesticide species and its concentration within one quick analysis. Advantageously, the method could be continuously improved by future data input

In one aspect, provided herein is a method for pesticide detection, comprising:
(a) contacting a sample with cells expressing acetylcholinesterase;
(b) contacting an acetylcholinesterase substrate with the cells; and
(c) detecting a reaction product resulted from the acetylcholinesterase substrate.

In another aspect, provided herein is a kit for pesticide detection, comprising: an acetylcholinesterase expressed on cells, and an acetylcholinesterase substrate.

Preferably, the acetylcholinesterase substrate may comprise acetylcholine, acetylthiocholine (ATCh), propionylthiocholine or acetyl-3-methylcholine.

Preferably, the method may further comprise the step of adding a fluorometric indicator to the reaction product.

Preferably, the fluorometric indicator may comprise 5,5′-dithio-bis-[2-nitrobenzoic acid](DTNB), dithiodinicotuic acid (DTNA), 2,2′-dithiodipyridine (2-PDS), hydroxylamine, choline oxidase coupled with the peroxidase/phenol/aminoantipyrine, Au—NP seeds in the presence of AuCl4, resorufin butyrate, indoxyl acetate, N-[4-(7-diethylamino-4-methylcoumarin-3-yl) phenyl]maleimide, 10-acetyl-3,7-dihydroxyphenoxazine (Amplex Red reagent), quantum dots (QDs), thiol green indicator or AbRed indicator.

Preferably, the reaction product may be 2-nitro-5-thiobenzoic acid (TNB) provided that the acetylcholinesterase substrate is DTNB.

Preferably, the method may further comprise the step of quantifying the pesticide by referencing to a standard curve of an inhibition of acetylcholinesterase activity versus a concentration of the pesticide.

Preferably, the acetylcholinesterase may be
(i) an acetylcholinesterase in full length, or
(ii) an acetylcholinesterase with deletion of the transmembrane domain TM and cytoplasmic domain (CTD).

More preferably, the acetylcholinesterase comprises amino acids of SEQ ID NO: 1, SEQ ID NO: 3, SEQ ID NO: 7, SEQ ID NO: 8, SEQ ID NO: 9, SEQ ID NO: 10, SEQ ID NO: 11, or SEQ ID NO: 12.

Preferably, the sample may be an agricultural product.

Preferably, the method may further comprise the step of grinding the agricultural product with pestles in Eppendorf tube or dipping the agricultural product with Q-tip. Alternatively, the method may further comprise a step of soaking the agricultural product into an extraction solution including water, PBS, DMSO, acetone, or ethanol.

Preferably, the pesticide may comprise compounds that phosphorylates or carbamoylate acetylcholinesterase.

More preferably, the pesticide may comprises organophosphates (OP) or carbamates (CB).

Preferably, the OP may comprise malathion, malaoxon, paraoxon, methyl parathion, naled, disulfoton, trichlorfon, triazophos, diazinon, dimethoate, phoxim, isoxathion, pyridaphenthion, pyraclofos, terbufos, profenofos, azinphos-methyl, isofenphos, pirimiphos-methyl, monocrotophos, temephos, isazofos, fenthion, quinalphos, heptenophos, phosmet, thiometon, chlorpyrifos, prothiofos, chlorfenvinphos, cyanophos, ethion, methidathion, mecarbam, oxydemeton-methyl, demeton-S-methyl, phosalone, methamidophos, formothion, phorate, phosphamidon, fenitrothion, acephate, omethoate, isothioate, vamidothion, phenthoate, or dicrotophos; and the CB may comprise carbosulfan, fenobucarb, pirimicarb, carbaryl, carbofuran, propoxur, butocarboxim, benfuracarb, bendiocarb, metolcarb, methomyl, thiofanox, thiodicarb, isoprocarb, XMC, xylylcarb, methiocarb, oxamyl, or formetanate.

In an embodiment, two or more acetylcholinesterases may be applied in the method or the kit, preferably by being independently displayed on cells in different wells.

Preferably, the method may further comprise the step of identifying the pesticide based on the reaction product of the two or more acetylcholinesterases.

More preferably, the two or more acetylcholinesterases may be different acetylcholinesterase mutants or acetylcholinesterases derived from different organisms. Alternatively, the acetylcholinesterases may be independently displayed in various expression level on cells in different wells.

Preferably, the kit may further comprise a fluorometric indicator. Optionally, the kit may further comprise a positive control comprising territrem B, donepezil hydrochloride, or cyclopenin.

Preferably, the acetylcholinesterase may be displayed on cell surface, baculovirus, or an occlusion body of baculovirus.

Preferably, the cell may be lyophilized. Preferably, the cell may be in a suspension, a tube, a chip or a plate. Optionally, the plate may be a single- or multi-well plate.

In one aspect, provided herein is a plasmid, comprising

a nucleotide sequence encoding:
(i) an acetylcholinesterase in full length, or
(ii) an acetylcholinesterase with deletion of the transmembrane domain TM and cytoplasmic domain (CTD) on cell surface;

hr1-hsp70 duel promoter;

p10 promoters; and

a honeybee mellitin signal peptide (HM) and/or a hexametric histidine tag (61H).

In one aspect, provided herein is a baculovirus, produced by a cell transfected with said plasmid.

In one aspect, provided herein is a cell, expressing
(i) an acetylcholinesterase in full length, or
(ii) an acetylcholinesterase with deletion of the transmembrane domain TM and cytoplasmic domain (CTD) on cell surface;
wherein the acetylcholinesterase comprises amino acids of SEQ ID NO: 1, SEQ ID NO: 3, SEQ ID NO: 7, SEQ ID NO: 8, SEQ ID NO: 9, SEQ ID NO: 10, SEQ ID NO: 11, or SEQ ID NO: 12.

In one aspect, provided herein is a method of preparing a cell for pesticide detection, comprising:

transfecting the cell with said plasmid, or infecting the cell with said baculovirus.

Preferably, the acetylcholinesterase may be from fruit fly, house fly, human, mouse, rat, fall armyworm, daphnia, chicken, moth, aphid, honey bee, shrimp, or fish.

Preferably, the nucleotide sequence may be derived from Drosophila melanogaster, Homo sapiens, Rattus norvegicus, Apis mellifera, Spodoptera frugiperda, Daphnia magna, or Bactrocera dorsalis.

Preferably, the nucleotide sequence may encode amino acids of SEQ ID NO: 1, SEQ ID NO: 3, SEQ ID NO: 7, SEQ ID NO: 8, SEQ ID NO: 9, SEQ ID NO: 10, SEQ ID NO: 11, or SEQ ID NO: 12.

Preferably, the nucleotide sequence may be SEQ ID NO: 2 which encodes the amino acid of SEQ ID NO: 1 or SEQ ID NO: 4 which encodes the amino acid of SEQ ID NO: 3.

Preferably, the plasmid may further comprise a reporter gene. More preferably, the reporter gene may comprise EGFP gene and a pag promoter.

Preferably, the plasmid may comprise a sequence of SEQ ID NO: 5 or SEQ ID NO: 6.

Preferably, the cell may be Spodoptera frugiperda IPLB-Sf21 (Sf21) cells.

Preferably, the baculovirus may be Autographa californica multiple nucleopolyhedrovirus (AcMNPV) or Bombyx mori nucleopolyhedrovirus (BmNPV).

Preferably, the AcMNPV is propagated in Spodoptera frugiperda IPLB-Sf21 (Sf21) cells.

Preferably, the cell is lepidopteran cells.

Preferably, the cell is Trichoplusia ni BTI-TN-5B1-4 High Five (Hi5) insect cell.

Preferably, a multiplicity of infection (VOI) for the AcMNPV is from 0.1 to 10, from 0.2 to 5, or from 0.5 to 2.

### BRIEF DESCRIPTION OF THE DRAWINGS

FIG. 1 illustrates construction of recombinant full length and fusing AChE by baculovirus expressing vector system. Schematic representation of the expression constructs used in the study. (a) AChE-FL contains the full-length AChE (Y408F) cDNA. (b) AChE-6MC contains the truncated AChE (Y408F), by which the TM and CTD of the AChE enzyme are deleted and replaced with those from baculovirus GP64 protein (i.e. 6MC). (c) The empty vector (pABEGhhp10) for generating the Control baculovirus (Cont-bac) without AChE expression. Ac-1250 bp and Ac-1433 bp: lateral sequences from the genome of baculovirus for homologous recombination to generate the recombinant virus. EGFP: EGFP reporter gene. pag:pag promoter. hr1-hsp70-p10: a duel promoter containing hr1-hsp70 and p10 promoters. HM: honeybee mellitin signal peptide. 6H: hexametric histidine tag.

FIG. 2 depicts determination of AChE activities for cell-surface expressed AChE recombinant proteins. Hi5 cells infected with vAChE-FL, vAChE-6MC and Cont-bac separately by an MOI of 0.5, and incubated 3 days. The activities of the AChE that was expressed in the infected cells were measured and evaluated by yellow colors. (a) Standard solution curve was established by the standard AChE solution (Abcam). Individual Cont-bac (b), vAChE-FL (c) and vAChE-6MC (d) clones were visualized in 96-well plates using substrate acetylthiocholine (ATCh) and Ellman's reagent DTNB solution.

FIG. 3 depicts evaluation of the lyophilization effect to cell-surface expressed AChE proteins. Hi5 cells infected with vAChE-FL (AChE-FL), vAChE-6MC (AChE-6MC) or Cont-bac (Cont), respectively with an MOI of 0.5 for 3 days, were subjected to lyophilization for 5 hours. Mock: Cells without virus infection. −lyo: cells without lyophilization. +lyo: cells with lyophilization. (a) Cell morphology examined by fluorescence and bright field microscope. (b) The AChE activity of the recombinant baculoviruses-infected cells was determined with and without the lyophilization.

FIG. 4 depicts optimization of recombinant baculovirus expressing AChE membrane proteins. (a) Visualization of AChE activity determination for the 96-well Hi5 cell samples separately infected with vAChE-FL (AChE-FL), vAChE-6MC (AChE-6MC) and Cont-bac (Cont) with various MOIs as indicated. All of the infection conditions were done in triplicate. (b) Quantification of AChE activities from the infected cells.

FIG. 5 depicts detection of OP and CB insecticide residues by cell-surface displayed AChEs. Hi5 cells were infected with AChE-FL-bac (FL), AChE-6MC-bac (6MC) or WT-bac (WT) respectively, for 3 days. After the removal of media, the cells were subjected to a 5-hour lyophilization in the 96-well plates. Two OP and two CB insecticide compounds were serial-diluted and added to the infected cell samples, as well as a 100 mU/mL AChE (ST, Abcam) in 96-well plates and the commercialized AChE (Sichen) in cuvette for a further comparison. AChE activities were analyzed for all these sample in order to determine the sensitivities of these AChE to different insecticides. DMSO with different concentrations in DPBS were added to determine the background effects. Pesticides tested are: (a) paraoxon ethyl, (b) malaoxon, (c) carbofuran (d) carbaryl. Regions covered by pink blocks showed that highest limit of residue pesticide allowance detectable from various agricultural products.

FIG. 6 depicts construction of recombinant AChEs from seven species for baculovirus expressing vector system. Schematic representation of the expression constructs for recombinant AChE from (a) Drosophila melanogaster (Y408F mutant), (b) Homo sapiens (Hs), (c) Rattus norvegicus (Rn), (d) Apis mellifera (Am), (e) Spodoptera frugiperda (Sf), (f) Daphnia magna (Dam), and (g) Bactrocera dorsalis (Bd). EGFP: EGFP reporter gene. pag: pag promoter. hr1-hsp70-p10: a dual promoter containing hr1-hsp70 and p10 promoters. HM: honeybee mellitin signal peptide. 6H: hexameric histidine tag.

FIG. 7 depicts detection of pesticides by multispecies AChEs. Hi5 cells were infected with vDmAChE (Dm), vHsAChE (Hs), vRnAChE (Rn), vAmAChE (Am), vSfAChE (Sf), DamAChE (Dam), and vBdAChE (Bd), respectively. After harvested at 3 days post infection, the cells were diluted in PBS into three different concentrations: 5×10³, 1.5×10⁴ and 4.5×10⁴ cells per well. Five pesticides, i.e., (a) chlorpyrifos, (b) ethion, (c) carbaryl, (d) carbofuran, and (e) methomyl, were serial-diluted from 10⁻³ to 10⁻⁹ M and added separately into the infected cells in 96-well plates. The remaining AChE activities were measured by adding substrate ATCh and DTNB solutions, and determined the optical density at 412 nm.

FIG. 8 depicts using machine learning to achieve fast identification of pesticide residues assayed by multispecies AChE. (a) The detection results from the multispecies AChE platform for each pesticide with specific concentration were collected and organized into a 21-parameter data set. (b) These data sets were used to train and build up the identification model. Once the data sets from an unknown pesticide determinants (c) input, the identification model could discriminate both pesticide and concentration (d).

### DETAILED DESCRIPTION

The foregoing and other aspects of the present disclosure will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process, or method.

The transitional phrase “consisting of” excludes any elements, steps, or ingredients not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

As used herein, the term “about” is used to indicate that a value includes for example, the inherent variation of error for a measuring device, the method being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive, although the disclosure supports a definition that refers to only alternatives and “and/or.”

Unless otherwise defined, all technical and scientific terms used herein have the same meaning as commonly understood by one of ordinary skill in the art to which this invention belongs. All publications, patent applications, patents and other references cited herein are incorporated by reference in their entireties for the teachings relevant to the sentence and/or paragraph in which the reference is presented.

As used herein, acetylcholinesterase (AChE) means a serine hydrolase in the α/β fold hydrolase protein superfamily which terminates nerve signals by catalyzing the hydrolysis of the neurotransmitter acetylcholine. In one embodiment, AChE is derived and optimized from Drosophila melanogaster (SEQ ID NO: 1), Homo sapiens (SEQ ID NO: 7), Rattus norvegicus (SEQ ID NO: 8), Apis mellifera (SEQ ID NO: 9), Spodoptera frugiperda (SEQ ID NO: 10), Daphnia magna (SEQ ID NO: 11), or Bactrocera dorsalis (SEQ ID NO: 12). In a related aspect, derivatives, fragments and variants of these AChE equivalents are also envisaged for the methods, kit, and plasmid of the present application.

In one embodiment, mutants can be prepared by site-directed mutagenesis (or evolution methods, see e.g., Devlin et al., Science (1990) 249:404-406; and Scott & Smith, Science (1990) 249:386-390) using a conventional oligonucleotide directed in vitro mutagenesis system such as that described by Eckstein et al., Nucleic Acids Research (1985) 13:8749-8785. (See also, U.S. Pat. No. 6,001,625). Other conventional PCR techniques known in the art may be used.

In one embodiment, the selection of residues for replacement is based on a molecular model, using the crystal structure of AChE published by Sussman, et al. on an Evans and Sutherland PS390 platform with Biosym software.

As used herein, “sample,” including grammatical variations thereof, means materials obtained from agricultural product. For example, such samples include, but are not limited to, a part of the agricultural product, e.g. skin, that is potentially contaminated by a pesticide or a insecticide.

Methods are described which distinguish between various classes of pesticides or insecticides, such as the compounds that phosphorylates or carbamoylate acetylcholinesterase, e.g. organophosphates (OP) or carbamates (CB), by analyzing the fluorescence or the color of reaction product of the present application.

In one embodiment, the AChE can be immobilized or lyophilized on a chip, a tube, or a plate to detect AChE inhibition. The plate may be a single- or multi-well plate.

The present application also provides kits. Such kits comprises an acetylcholinesterase expressed on cells, and an acetylcholinesterase substrate. The acetylcholinesterase expressed on cells may be in a suspension, a tube, a plate, a glass vial or jar, a plastic pack, etc. In one embodiment, the acetylcholinesterase expressed on cells may be in a suspension, a tube, a chip or a plate. The acetylcholinesterase expressed on cells may be lyophilized and contained in a microtiter plate or a chip. For example, a method of immobilization may include, but is not limited to, the method as described in Taylor et al. (U.S. Pat. No. 5,192,507). In other embodiments, the kit may comprise a container of a plastic, glass, or metal tube which contains the enzyme, and the tube may possess an inlet means at one end and an outlet means at the other end.

The kit may further comprise a negative control sample. Such a negative control sample will contain no pesticides, insecticides or the like or a very low amount of pesticides, insecticides or the like. The kit may also comprise a positive control sample, which will comprise, typically, an amount of pesticides, insecticides or the like which is equal to or greater than the amount of pesticides, insecticides or the like, e.g. territrem B, donepezil hydrochloride, or cyclopenin, which is considered a positive result. The kit may also contain chemicals, such as buffers or diluents, and sample handling means, such as pipettes, reaction vials, vessels, tubes, or filters.

In addition, the kit may comprise written instructions on a separate paper, or any of the container means, or any other packaging. These instructions will usually set forth the conditions for carrying out the detection method, such as mixing ratios, amounts, incubation times, etc., and criteria for evaluating the results of the method, including spectra charts.

In an embodiment, two or more sets of cells expressing acetylcholinesterase are applied to detecting pesticides and the two or more sets of cell will generate different amount of reaction product upon contacting a sample.

In an aspect, the two or more sets of cells are constructed with different baculovirus clones so as to express different amount of AChEs in the two or more sets of cells. For example, one set of cell is constructed with baculovirus A clone and the other set of cell is constructed with baculovirus B clone, and the set of cell constructed with baculovirus A expresses more AChEs than the set of cell constructed with baculovirus B.

In another aspect, the two or more sets of cells are constructed with different AChEs so as to express different AChEs in the two or more sets of cells. For example, the different AChEs may be sensitive or insensitive AChEs, or the different AChEs may come from different origins, e.g. from vertebrates or invertebrates, or from fruit fly, house fly, human, mouse, rat, fall armyworm, daphnia, chicken, moth, aphid, honey bee, shrimp, or fish.

In a preferred embodiment, one set of cell is constructed with baculovirus A clone and the other set of cell is constructed with baculovirus B clone, in which the set of cell constructed with baculovirus A expresses more AChEs than the set of cell constructed with baculovirus B. In one embodiment, there are three scenarios when the two sets of cells contact the sample: (1) both the acetylcholinesterase activities in the set of cell constructed with baculovirus A and the set of cell constructed with baculovirus B are not inhibited by the sample, indicating the sample is not contaminated by the pesticide; (2) the acetylcholinesterase activities in the set of cell constructed with baculovirus A is not inhibited but the acetylcholinesterase activities in the set of cell constructed with baculovirus B is inhibited, indicating that the pesticide in the sample is below the permitted level; and (3) both the acetylcholinesterase activities in the set of cell constructed with baculovirus A and the set of cell constructed with baculovirus B are inhibited by the sample, indicating the sample is contaminated by the pesticide and above the permitted level.

In one embodiment, two or more AChE may be independently displayed on different cells in different wells to detect, quantify or identify pesticides. For example, cells with various acetylcholinesterase expressions may be applied to show stronger and weaker reaction colors to reflect pesticide concentrations so that the pesticides in different concentrations may be measured by AChE with different activities or expressions.

As an exemplary embodiment, cells with 1, 2, 3, 4 and 5 unit(s) of AChE may be applied for detection of pesticides or insecticides. By applying the Ellman's method, the detection of yellow color corresponds to no presence of OP or CB insecticides or the concentration thereof is below detection limit. As shown in the table below, the pesticides or insecticides were detected by 2 units of AChE but not 3 units of AChE. That is, the concentration of the pesticides or the insecticides may be semi-quantified with reference to a standard curve made by corresponding the unit of the AChE and the concentration of a specific pesticides or the insecticides. For instance, the concentration of the pesticide A is higher than or equal to 1.4×10⁻⁷M but lower than 1.6×10⁻⁷.

| | | Corresponding |
|---|---|---|
| | | concentration of the |
| Unit | Result (color) | pesticide A (M) |
| 1 | Clear | 1.2 × 10⁻⁷ |
| 2 | Clear | 1.4 × 10⁻⁷ |
| 3 | Yellow | 1.6 × 10⁻⁷ |
| 4 | Yellow | 1.8 × 10⁻⁷ |
| 5 | Yellow | 2.0 × 10⁻⁷ |

Alternatively, different AChE mutants or AChE derived from different organisms may be applied to the method or kit above. In other words, AChE with various sensitivities may be displayed on different wells. Thus, the pesticides may be detected, quantified or identified by a series of AChE panel as discussed above.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

### Examples

Materials and Methods

Chemicals and Reagents

Acetylcholinesterase Assay Kit (standard AChE) was purchased from Abcam. Rapid Bioassay of Pesticides Residues Reagent Kit (commercial AChE) was purchased from Sichen.

Organophosphorus compounds (paraoxon ethyl, malaoxon, chlorpyrifos and ethion) and carbamate compounds (carbaryl, carbofuran and methomyl) for inhibition assays were purchased from Sigma-Aldrich. All four compounds were prepared as 1 mM stock solutions in dimethyl sulfoxide (DMSO) (Ameresco) prior to use. Different concentrations of each compound solution in assays were further prepared by serial dilution in DMSO, with final DMSO concentrations maintained below 2%. Dulbecco's phosphate-buffered saline (DPBS) was purchased from Corning. All the chemicals used were of analytical grade.

Cell Lines and Viruses

Spodoptera frugiperda IPLB-Sf21 (Sf21) cells for generating recombinant baculovirus (AcMNPV) were grown at 26° C. in TC100 insect medium (US Biological) containing 10% fetal bovine serum (FBS). Trichoplusia ni BTI-TN-5B1-4 High Five (Hi5) cells for maximum recombinant protein expression were grown at 26° C. in ESF 921™ insect cell culture medium (Expression Systems). Recombinant baculoviruses, including AChE-FL, AChE-6MC or control virus without AChE cDNA (WT-bac), were generated by co-transfecting the FlashBAC™-modified AcMNPV genome (Mirus) with transfer vector plasmids into Sf21 cells using TransIT®-Insect Transfection Reagent (Mirus). Recombinants were propagated and isolated by serial dilution. The titers of the virus clones were determined by both quantitative PCR (qPCR) and 50% tissue culture infective doses (TCID 50).

Construction of Plasmids

AChE sequence was derived from Drosophila melanogaster [10] with a mutation Y408F[11], which sequence was synthesized by Bio Basic Inc. Plasmid pABEGhhp10 is a transfer vector with the amino-terminal honeybee mellitin signal peptide and the carboxyl-terminal GP64 transmembrane and CTD domain (6MC). Plasmid pABEGhhp10 carrying the pag promoter to drive EGFP fluorescence protein expression and allowing insertion of foreign sequences downstream of p10 promoter. AChE Y408F sequence was amplified in a polymerase chain reaction using KOD Hot Start Master Mix (Merck) and two specific oligonucleotide primers. The primers used for amplifying FL coding region insert fragment without 6MC were HAChEifp (5′-CACCATCACCATCACGTCATCGATCGCCTGGTT-3′, SEQ ID NO: 13) and AChE2rp (5′-CGGATCAATTAATTAGAACACGCGCTTAGTTCTC-3′, SEQ ID NO: 14). The primers used for amplifying C-terminal GPI-anchoring truncated AChE 6MC coding region insert fragment were HAChEifp (5′-CACCATCACCATCACGTCATCGATCGCCTGGTT-3′, SEQ ID NO: 13) and AChEdeGPIrp (5′-ATGACCAAACATGAAATCTCCGTCACATGTGCC-3′, SEQ ID NO: 15). Plasmid pABEGhhp10 was amplified in a polymerase chain reaction using KOD Hot Start Master Mix (Merck) and two specific oligonucleotide primers. The primers used for amplifying FL coding region vector fragment were pABhhp10HM6H2fp (5′-ACTAAGCGCGTGTTCTAATTAATTGATCCGGGTTATTAGTACATTTAT-3′, SEQ ID NO: 16) and HAChEvrp (5′-CAGGCGATCGATGACGTGATGGTGATGGTGATGC-3′, SEQ ID NO: 17). The primers used for amplifying 6MC coding region vector fragment were pABhhp10HM6Hfp (5′-ACATGTGACGGAGATTTCATGTTTGGTCATGTAGTTAACTTTGT-3′, SEQ ID NO: 18) and HAChEvrp (5′-AGGCGATCGATGACGTGATGGTGATGGTGATGC-3′, SEQ ID NO: 17). The insert fragments were fused with vector fragment by using In-fusion HD Cloning Kits (Takara Bio USA, Inc.). After transformation and picking up colonies, plasmids were amplified, extracted and verified by sequence analysis. The resulted plasmids containing AChE-full length, AChE-6MC, and control virus without AChE insertion are abbreviated as FL, 6MC and Cont, respectively. The resulted plasmids were named as pAChE-FL (SEQ ID NO: 5), pAChE-6MC (SEQ ID NO: 6), and pCont-Bac; and the resulted recombinant baculoviruses were named as vAChE-FL, vAChE-6MC, and vCont-Bac. In an embodiment, the AChE-FL comprises amino acids of SEQ ID NO: 1. In a preferred embodiment, the AChE-FL is encoded by the nucleotide sequence of SEQ ID NO: 2. In another embodiment, the AChE-6MC comprises amino acids of SEQ ID NO: 3. In another preferred embodiment, the AChE-6MC is encoded by the nucleotide sequence of SEQ ID NO: 4.

In addition, AChE sequence derived from Homo sapiens (HsAChE), Rattus norvegicus (RnAChE), Apis mellifera (AmAChE), Spodoptera frugiperda (SfAChE), Daphnia magna (DamAChE), and Bactrocera dorsalis (BdAChE) were synthesized by Bio Basic Inc. To generate the transfer vectors for recombinant baculoviruses, all above mentioned AChE sequences were cloned into vector, pABEGhhp10. The resulted recombinant baculoviruses were designated as vHsAChE, vRnAChE, vAmAChE, vSfAChE, vDamAChE, and vBdAChE, respectively; the resulted recombinant plasmid were designated as pHsAChE, pRnAChE, pAmAChE, pSfAChE, pDamAChE, and pBdAChE, respectively. pAChE-FL was used here to express the AChE, DmAChE. In an embodiment, HsAChE comprises amino acids of SEQ ID NO: 7. RnAChE comprises amino acids of SEQ ID NO: 8. AmAChE comprises amino acids of SEQ ID NO: 9. SfAChE comprises amino acids of SEQ ID NO: 10. DamAChE comprises amino acids of SEQ ID NO: 11. BdAChE comprises amino acids of SEQ ID NO: 12.

Expression of AChEs and Collection of AChE-Displayed Cells

The primary viral stock (V₀) was obtained from transfection of Sf21 cells with flashBAC™ ULTRA (Mirus Bio) and DNA-Cellfectin® (Invitrogen) mixture, and the titer of single virus (V₁) was improved by a serial dilution of the stock viral solution and isolated by plaque purification. The propagated single virus (V₂) was amplified with V₁ virus infection of Sf21 cells. Hi5 cells (4×10⁵ cells/mL) in 96-well microplates were infected with the V₂ virus and grown at 26° C. for 3-4 days. Hi5 cells were infected with a multiplicity of infection (MOI) of 0.1-10, preferably 1-5, 0.2-5, or 0.5-2. For instance, the MOI may be 0.1, 0.2, 0.3, 0.4, 0.5, 0.6, 0.7, 0.8, 0.9, 1, 2, 3, 4, 5, 6, 7, 8, 9 or 10. To screen out recombinant baculovirus expressing AChE by using fluorescence as a biomarker, e.g. the expression of the EGFP reporter. The expression of AChEs was determined by AChE activity. After 3 days of infection, the infected cells were scraped from culture plates or flasks by cell scrapers and suspended in DPBS. Cell numbers were counted by a cell counter and diluted into several different concentrations. In a preferred embodiment, the final concentrations used in 96-well plate assay for a kind of AChE-displayed cell could be 5×10³, 1.5×10⁴, and 4.5×10⁵ cells/well. Furthermore, for collection of AChE-displayed cells, medium was removed carefully from 96-well microplates. Infected cells in microplates underwent lyophilization process to remove remaining moisture for 5 hours. Dried microplates were sealed and preserved at 4° C.

AChE Activity and Inhibition Assays

AChE activity was determined by Ellman's method. For each 96-well, 50 μl of 3 mM ATCh and 50 μl of 3 mM Ellman's reagent DTNB solution was added. After 10 minutes of incubation at room temperature, average absorbance at 412 nm was determined. To determine inhibition of AChE activity by each pesticide, stock solutions of pesticides, such as chlorpyrifos, ethion, carbaryl, carbofuran and methomyl, were diluted into concentrations ranging from 10⁻³ M to 10⁻⁹ M in DPBS with 1% DMSO. The diluted solutions were then added individually into 96-well microplates with Hi5 cells infected by the seven recombinant viruses (vDmAChE, vHsAChE, vRnAChE, vAmAChE, vSfAChE, DamAChE, and vBdAChE). After incubation for 10 minutes, substrate ATCh and DTNB solution were added to the reaction mixture. After 10 minutes of incubation at room temperature, residual activity was determined with a microplate reader at 412 nm.

The acetylcholinesterase stock solution was diluted into 50 units/mL in assay buffer at ratio 1:50 to generate 1000 mU/mL acetylcholinesterase standard solution. Then, the 1000 mU/mL acetylcholinesterase standard solution was further diluted into 300, 100, 30, 10, 3, 1 and 0 mU/mL for determining the average absorbance of each standard. The reading of each standard was plotted as a function of the amount of acetylcholinesterase to establish a standard curve, and the corresponding amount of AChE in each test samples was calculated by the derived linear equation. To calculate the inhibition of AChEs activity (%), vCont-Bac infected cells (without expression of AChE on the surface) served as the negative control, whereas Abcam purified protein, Sichen commercial protein, vAChE-FL and vAChE-6MC infected cells without pesticide incubation served as the positive control. The activity of the positive control was considered 100%.

The remaining activity of AChE is evaluated by the following equation:

Inhibition of AChEs activity (%)(ΔA₀−ΔA_{S})/ΔA₀)×100

In which, ΔA₀ indicates the change of absorbance of the positive control, and ΔA_{S} indicates the change of absorbance of the applied pesticide incubated solution. The value of IC₅₀ (concentration required to inhibit activity by 50%) of AChEs was calculated according to the linear equation established by inhibition activity curve[12].

Machine Learning Using AChE Activities Determined from Multispecies AChEs

An Extreme Gradient Boosting (XGBoost) model was developed for the discrimination of pesticide and concentration applied to the multispecies AChE detection platform. To train and test the XGBoost model, the optical density data from multispecies AChE detection for each pesticide with specific concentration was organized into a 21-parameter data set, including readings from seven AChEs and three cell concentrations. A total of 120 data sets from three experimental repeats were split into training (106) and test (14) sets. The 106 training sets were used to build up the model in Python using scikit-learn. The outcome model was confirmed using test data sets and evaluated the identification accuracy.

Results

Highly Sensitive AChE Mutant and Seven AChEs from Different Species were Synthesized for Insect Cell Surface Expression

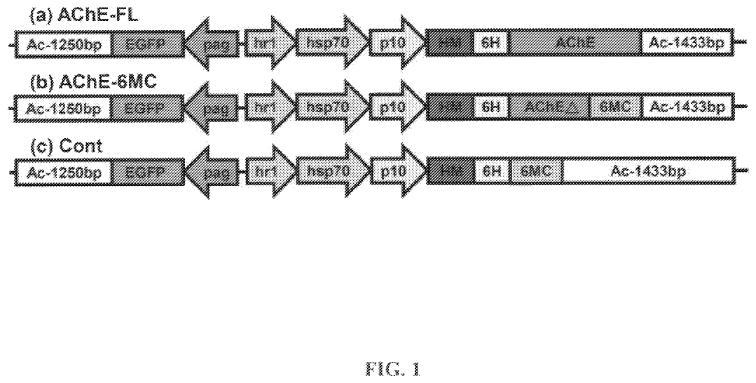

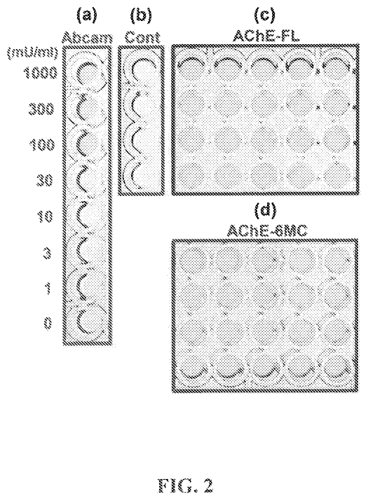

The AChE from D. melanogaster chosen to be the enzyme in the present application, and synthesized the oligonucleotides encoding this enzyme with codon optimization for baculovirus-insect system. To increase the enzyme sensitivity, Y408F mutation was introduced the synthesized gene, which has showed to increase enzyme sensitivity up to 12 folds[11]. Two constructs were generated to express this recombinant enzyme (FIG. 1). The construct pAChE-FL comprises the full-length AChE (Y408F), an N-terminal honeybee mellitin signal peptide (HM) for protein secretion, and a hexametric histidine tag (61-H), all of these are placed under a duel promoter containing hr1-hsp70 (i.e. hsp70 promoter fusing and hr1 enhancer sequence), and p10 promoter (FIG. 1, part (a)). The other construct, pAChE-6MC, is generated with deletion of the transmembrane (TM) and cytoplasmic domain (CTD) of the enzyme. The AChE-6MC (FIG. 1, part (b)) is instead fused with the TM and CTD from baculovirus GP64 protein, and placed under the same expression vector pABEGhhp10 (FIG. 1, part (c)). The replacement of GP64 TM and CTD have been reported to improve the anchor of recombinant protein to the envelope of the baculovirus and surface of the insect cells¹²⁻¹⁴.

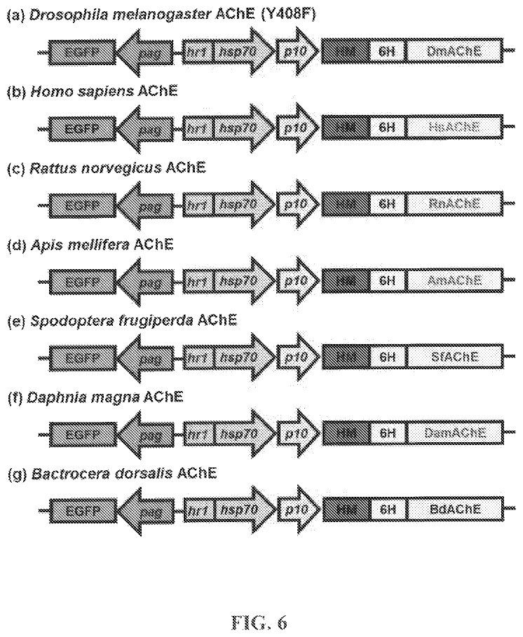

Other than the D. melanogaster Y408 mutants, the AChEs from six different species (FIG. 6) were chosen to be the multiple enzymes in the present application. Herein, for a clear distinction, the D. melanogaster Y408 mutants was designated as DmAChE (FIG. 6, part (a)) in the embodiment of multiple enzymes. The other six AChEs were named by the abbreviations of their species name, including HsAChE, RnAChE, AmAChE, SfAChE, DamAChE, and BdAChE. Oligonucleotides encoding these six enzymes were synthesized with codon optimization for the baculovirus-insect system and cloned into the vector, pABEGhhp10, using the same strategy for AChE-FL.

Insect Cell Surface-Expressed AChE Exhibited Enzyme Activity

Two recombinant baculoviruses vAChE-FL and vAChE-6MC were generated to respectively to express AChE-FL and AChE-6MC. The empty vector, pABEGhhp10, was also used to generate recombinant virus, designated as the Cont-bac, as a negative control (FIG. 1, part (c)). High Five (Hi5) insect cells were used for enzyme expression because Hi5 cells could express higher level of recombinant protein among the commonly used insect cells. Furthermore, the cells have been adapted in serum-free medium, so the final enzyme reaction could be performed without the serum interference. Each of 20 single viruses was selected for vAChE-FL-bac and vAChE-6MC-bac, and four for Cont-bac, to infect the Hi5 cells with an MOI of 0.5 in 96-well plate, and assayed their AChE activities at 3 days post infection (dpi). A standard AChE solution was diluted into a range of 0-1000 mU/mL to establish the standard curve for enzyme activity (FIG. 2, part (a)). As the Cont-bac infected cells showed nearly transparent color after reacting with the substrate acetylcholine and coloring agent DTNB solution (FIG. 2, part (b)), cells infected with vAChE-FL (FIG. 2, part (c)) and vAChE-6MC (FIG. 2, part (d)) single viruses all showed visible yellow color, converting to an average of 60-80 mU/mL enzyme activity, respectively. One single virus showing the highest activity for each constructs was selected for the following experiments.

The Cell-Surface Expressed AChE Sustained the Lyophilization Process

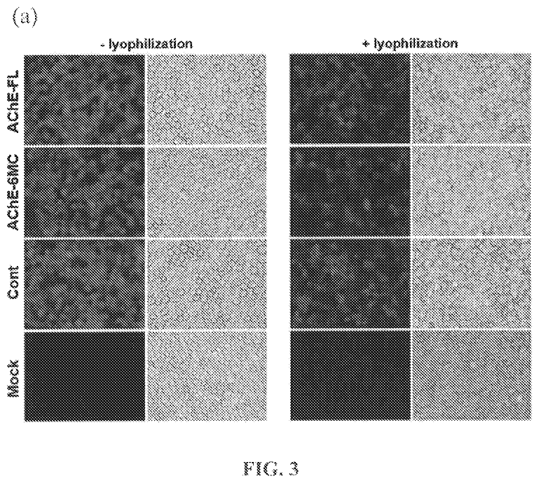

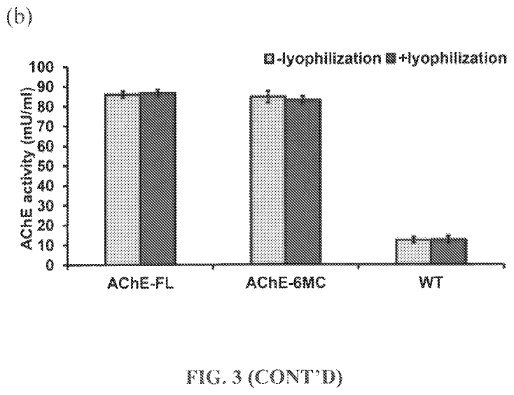

Although the AChE displayed on cell surface exhibited high enzymatic activity, cells containing medium is not convenience for transportation. In order to resolve this problem, medium was removed and the cells which still attaches to the wells were subjected to the lyophilization process. To know whether our cell surface-expressed AChE can sustain the freeze-vacuum drying process, Hi5 cells were infected with viruses expressing AChE-FL, AChE-6MC or Cont-Bac, respectively, with MOIs of 0.5 for 3 days, and then the cell samples on the 96-well plate were subjected to lyophilization. The cell morphology was observed both before and after lyophilization by microscopy. Since all of the three viral constructs, vAChE-FL, vAChE-6MC, and Cont-Bac, contain a pag promoter to drive EGFP reporter gene, cells infected by these three viruses exhibit green fluorescence, compared to the Mock-infected cells showed no fluorescence (FIG. 3, part (a)). After the lyophilization, all the cell samples showed a shrinkage cell morphology, but these three infected cell still preserved strong green fluorescence (FIG. 3, part (a)). The AChE activity for these cell samples were analyzed, and found that the freeze-vacuum drying process did not disrupt the enzymatic activities for AChE-FL and AChE-6MC, nor increase the background reads of Cont-Bac (FIG. 3, part (b)).

A High AChE Activity could Still be Achieved by Virus Infection with Low MOI.

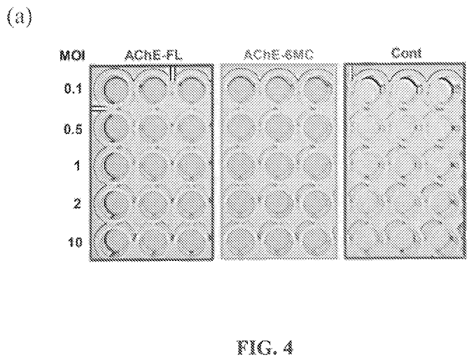

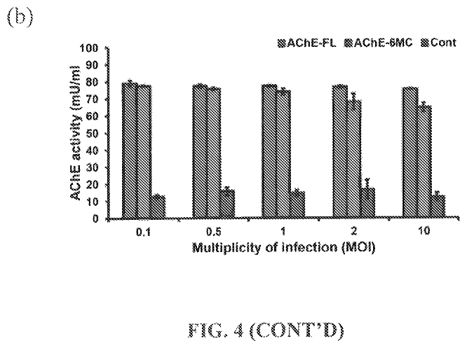

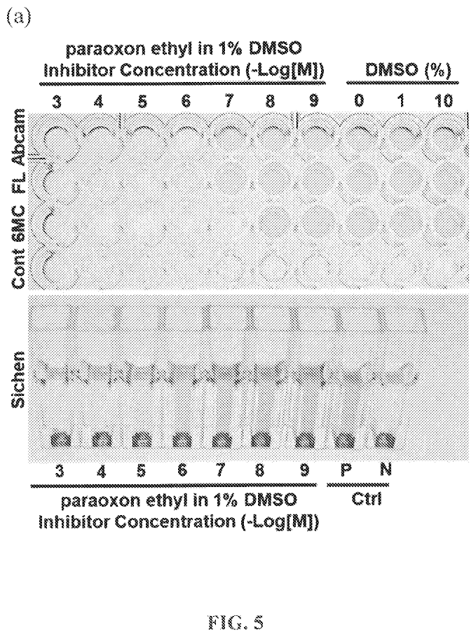

After determining the lyophilization condition, we aimed to optimize the virus infection conditions for our AChE-expressing cell samples. Hi5 cells were infected separately by recombinant virus vAChE-FL, vAChE-6MC and Cont-Bac, with MOI of 0.1, 0.5, 1, 2, and 10 in triplicate. At 3 dpi, all the infected cell samples were subjected to lyophilization and then the activity of AChE was determined (FIG. 4, part (a)). While all of the cell samples infected by vAChE-FL showed slightly higher AChE activity than those infected by vAChE-6MC, the infected cells for both viruses showed a trend that AChE activity decreased with the increasing MOIs, this is especially true for the cells infected by vAChE-6MC (FIG. 4, part (b)). For Cont-bac infected cells, all of the determined MOIs showed no significant difference in activity (FIG. 4, part (b)). Based on these results, MOI=0.1 was used as the standard infection condition for the display of AChE.

Cell-Surface Display of AChE-FL and AChE-6MC can be a Convenient Platform to Detect the OP and CB Insecticides

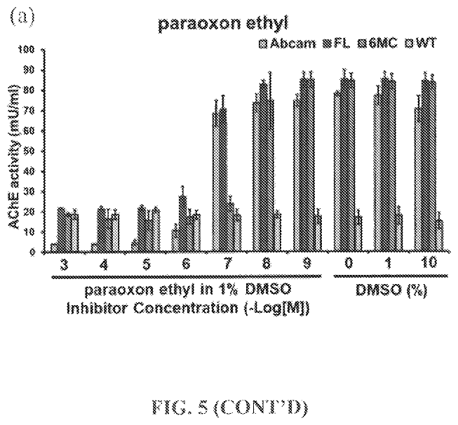

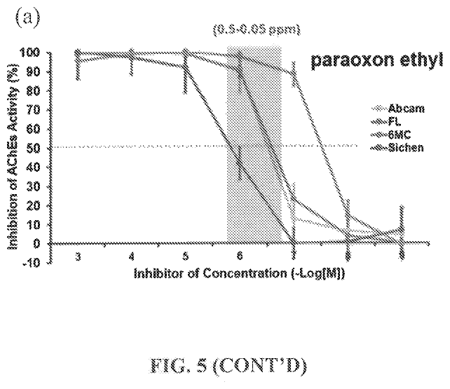

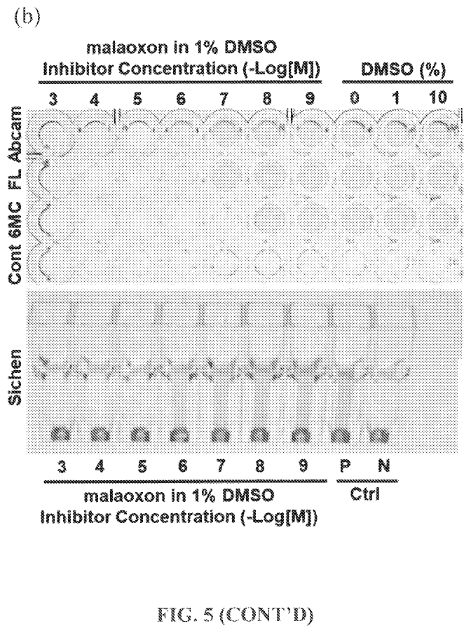

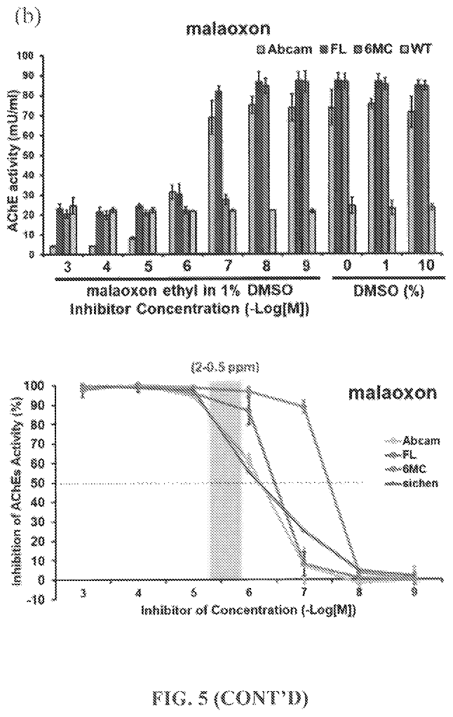

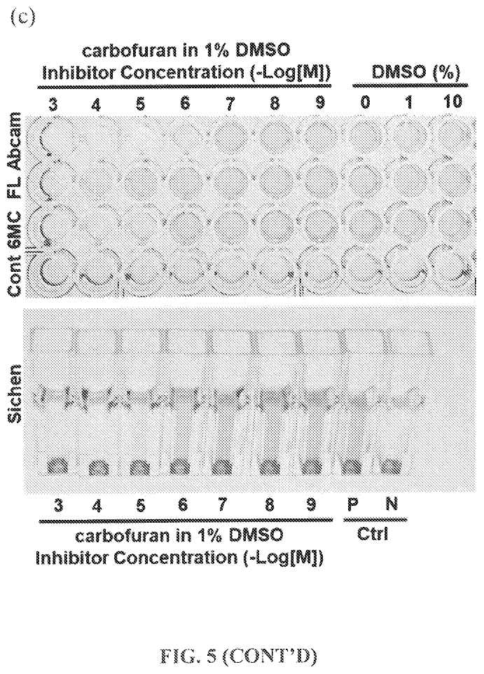

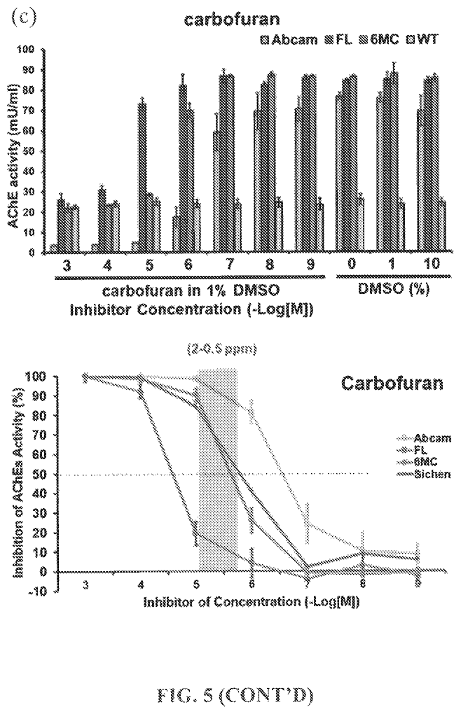

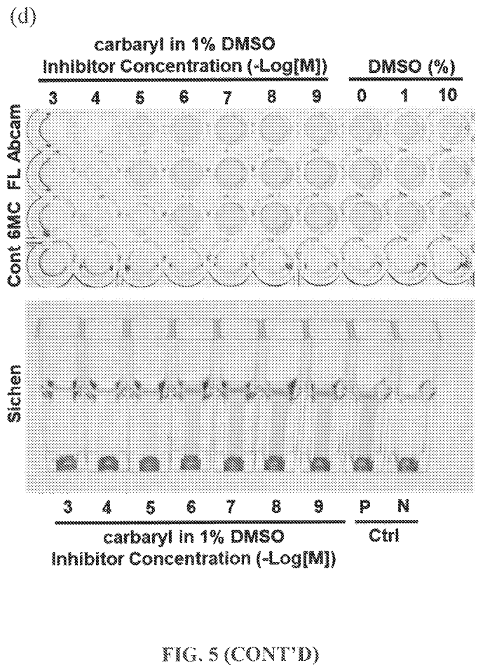

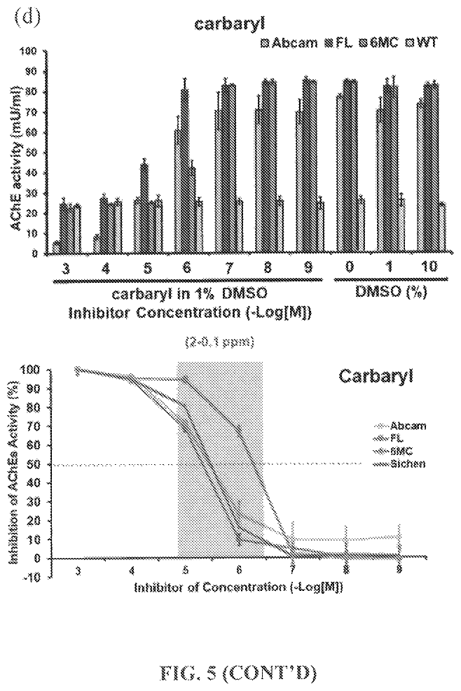

To evaluate the potential of the cell-based AChEs according to an embodiment of the present application to determine the OP and CB insecticides, two OP (e.g. paraoxon ethyl and malaoxon) and two CB (e.g. carbofuran and carbaryl) insecticide compounds were tested, in our system and compared the detection ability with the purified AChE (Abcam) as a standard (FIG. 5). Cell-surface display of AChE by recombinant baculoviruses vAChE-FL and vAChE-6MC showed they can properly display of AChE on the cells for convenient to detect the OP and CB insecticides with precise and ease. As for a comparison, the test results using the most common AChE insecticide diagnostic kit in Taiwan sold by Sichen Inc. were also included, and the IC50 for all of the test samples were also calculated (Table 1). For Sichen, since they acquire the AChE from fly, also they have to use cuvette (1 mL) for measurement, thus high quantity of AChE should be used. Furthermore, they suggest to measure the color reaction using spectrophotometer. In the present application, however, there are no need for fly rearing and no need for AChE purification. 96-well plate was applied, which requires only 100 μL solution for detection, and easily to handle, and can be read by bare eye for the detection of residue pesticides.

TABLE 1
IC₅₀ of OP and CB towards AChEs (Unit: M)

| | AChE-FL | AChE-6MC | Abcam | Sichen |
|---|---|---|---|---|
| Paraoxon ethyl | 4.9 × 10⁻⁷ | 5.5 × 10⁻⁸ | 5.1 × 10⁻⁷ | 2.3 × 10⁻⁶ |
| Malaoxon | 5.6 × 10⁻⁷ | 6.2 × 10⁻⁸ | 8.2 × 10⁻⁷ | 9.5 × 10⁻⁷ |
| Carbofuran | 4.8 × 10⁻⁵ | 4.5 × 10⁻⁶ | 5.4 × 10⁻⁷ | 2.8 × 10⁻⁶ |
| Carbaryl | 6.8 × 10⁻⁶ | 6.5 × 10⁻⁷ | 6.5 × 10⁻⁶ | 5.9 × 10⁻⁶ |

In conclusion, a cell-based detection system have been developed for convenient examination of insecticides residue in agricultural products. This is a novel technology, to replace conventional methods without tedious fly rearing, acetylcholinesterase purification, cuvette assay, and spectrophotometer determination, for better detection of insecticide residues. Thus, disclosure herein provides a convenient and rapid solution for the detection of OP and CB insecticides, which are difficult to achieve previously. Since AChE-FL and AChE-6MC showed roughly one log differences in the detection of different residual pesticides, the combination of these two should cover the ranges of residual pesticides transition from overdose to the range of acceptable, and provide an unprecedented easy system for better reading and detection.

The Multispecies AChE Platform Exhibited Distinguished Sensitivities to Various OP and CB Pesticides

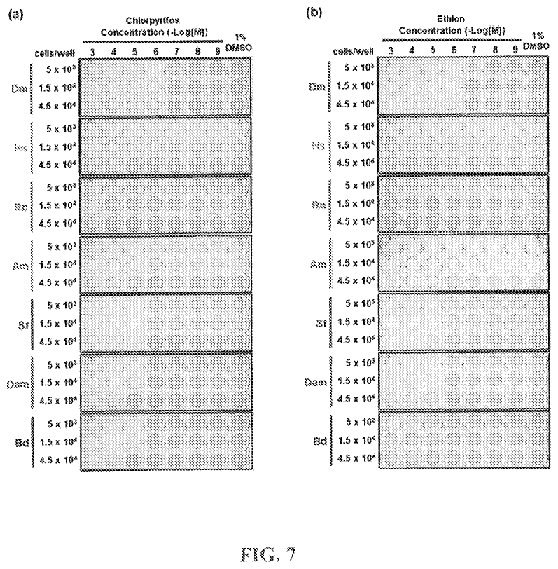

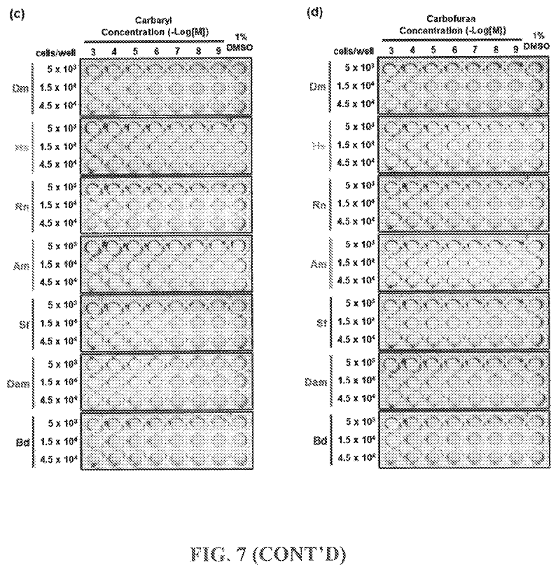

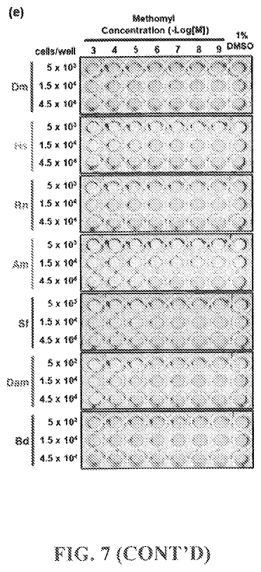

After generation of the recombinant baculoviruses for the six AChEs (vHsAChE, vRnAChE, vAmAChE, vSfAChE, DamAChE, and vBdAChE), the Hi5 cells were infected with vDmAChE and these viruses, respectively. After 3 days of infection, the infected cells were scraped from culture plates or flasks by cell scrapers and suspended in DPBS. To adjust the concentration of AChE enzymes, we altered the cell numbers added in each 96-well. The initial cell numbers were counted by a cell counter and three different cell concentrations were adjusted by dilution or concentration. In FIG. 7, the results of using cell concentrations of 5×10³, 1.5×10⁴, and 4.5×10⁴ cells/well were showed. After adding the pesticides, the inhibition of AChE activity would result in a decrease in yellow coloring which can be determined by the absorbance at 412 nm. The seven AChE with different initial concentrations exhibited various sensitivities to the OP including chlorpyrifos and ethion, and CB including carbaryl, carbofuran, and methomyl pesticides (FIG. 7).

Machine Learning Approach Enables the Fast Discrimination of Pesticide Residues Assayed by Multispecies AChE

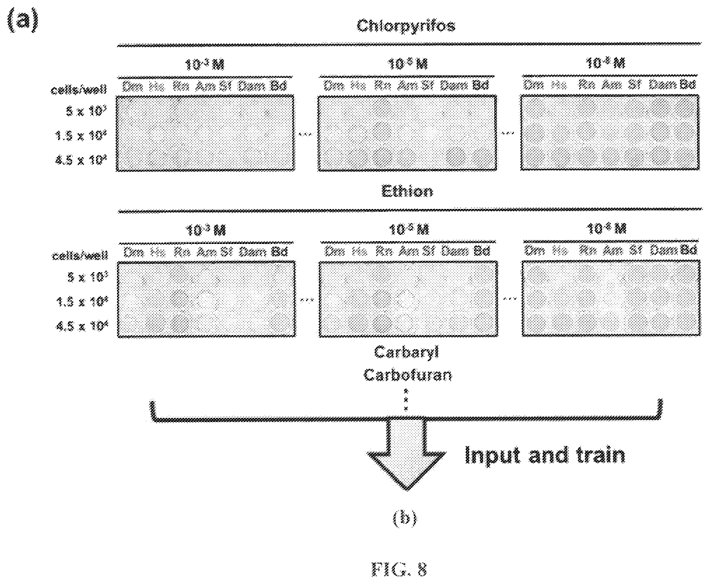

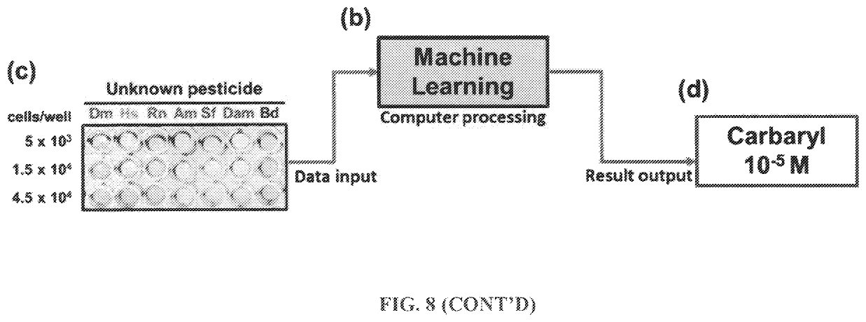

Since the multispecies AChEs showed different sensitivities to the tested pesticides, the outcome optical density data for a pesticide with specific concentration could become an identification panel including 21 readings from seven AChEs and three cell concentrations (FIG. 8, part (a)). We used a total of 120 data sets from three experimental repeats to develop a pesticide identification model. To train the model built by XGBoost, 106 training sets were input into the algorithm and 14 test sets were used to evaluate the identification accuracy (FIG. 8, part (b)). The model could be continuously trained by inputting more determinants and improving accuracy.

In conclusion, a method and kit for novel multispecies AChE pesticide determination have been developed, which can discriminate both residual pesticide and concentration. The combination of machine learning enables the fast identification of specific pesticide with concentration and the system could be further optimized by adding more training data.

Discussion

OP and CB insecticides are common chemical pesticides applied onto agricultural cultivars, which frequently results crop contamination, and threatening human health. In the present application, by displaying of an AChE mutant highly sensitive to OP and CB insecticides on cell surface using baculovirus, a novel determination platform for the convenient detection of these insecticides have been developed with or without the need of spectrophotometer to assist measurement. Several conditions were also examined to optimize the system, such as the MOI for virus infection and lyophilization. Remarkably, the replacement of GP64 TM and CTD were found to improve surface-displayed AChE sensitivity to the insecticide residues.

In addition to natural substrates acetylcholine, AChE can also be used to hydrolyze many other esters, including esters of thiocholine such as acetylthiocholine (ATCh), propionylthiocholine and acetyl-3-methylcholine¹⁵. AChE is inhibited by acetylcholine, choline, eserine, quinidine, tetramethyl ammonium ions, p-carboxyphenyltrimethylammonium iodide, trimethyl (p-ami-nophenyl) ammonium chloride hydrochloride, neostigmine, ethionamide, dimethoate, phosphatidylserine, prostigmine, ammonium salts, and various organophosphorus, orga-nochlorine, and carbamate pesticides. Several spectrometric-based assay have been widely used including UV-Vis assays, fluorometric assays and mass spectrometric assays¹⁶. The most prevalent method used in the study is Ellman method. AChE catalytically hydrolyzes ATCh to produce thiocholine, which reacts with DTNB to produce yellow TNB which can be measured at 412 nm. An alternative method is using dithiodinicotnic acid (DTNA) or 2,2′-dithiodipyridine (2-PDS) to replace DTNB. The products of the reaction can be measured at 344 nm¹⁷. Apart from Ellman method, other assays of AChE activity and inhibition could also be applied to our surface displayed detection system describe as following. A colorimetric assay based on detecting substrate acetylcholine reacts with hydroxylamine to yield acetylhydroxamic acid bearing Fe³⁺ that can be photometrically monitored¹⁸. Choline can be also detected by using choline oxidase coupled with the peroxidase/phenol/aminoantipyrine system, which produce a pink product with a maximum absorbance at 500 nm¹⁹. A nanotechnology-based sensing method by use of thiocholine to stimulate the catalytic enlargement of Au NP seeds in the presence of AuCl4 thus give rise to blue color with a maximum absorbance at 570 nm²⁰. Two fluorogenic substrates, resorufin butyrate and indoxyl acetate, are nonfluorescent compounds which can be hydrolyzed by cholinesterase to highly fluorescent materials with maximum absorbances at 580 nm and 470 nm respectively²¹. fluorogenic compound N-[4-(7-diethylamino-4-methylcoumarin-3-yl) phe-nyl] maleimide can also reacts with thiocholine to yield an intensely blue fluorescent product with a fluorescence emission at 473 nm²². AChE converts the acetylcholine to choline, followed by choline being oxidized by ChO to betaine and H₂O₂. In the presence of horseradish peroxidase, the latter reacts with a fluorogenic probe, the Amplex Red reagent (10-acetyl-3,7-dihydroxyphenoxazine) to generate the highly fluorescent product resorufin with a fluorescence emission at 571 nm and 585 nm (Molecular Probes, Inc.). A semiconductor nanoparticles, quantum dots (QDs), have unique fluorescence properties which can also reacts with H₂O₂ which quenches the QD luminescence. Their size-controlled fluorescence properties and high fluorescence quantum yields makes them superior optical labels for biosensing with a fluorescence emission at 570 nm²³. Two acetylcholinesterase assay kits with fluorometric thiol green indicator or AbRed indicator are designed for detection of AChE activity with a fluorescence emission Ex/Em=490/520 nm and 540/590 nm, respectively (Abcam, Inc.).

The ability of the surface-display AChEs to sense the insecticide residues according the present application were determined. The IC₅₀ for paraoxon ethyl, malaoxon, carbofuran, and carbaryl of the AChE-FL was 4.9×10⁻⁷, 5.6×10⁻⁷, 4.8×10⁻⁵, 6.8×10⁻⁶ M, respectively, and of the AChE-6MC was 5.5×10⁻⁸, 6.2×10⁻⁸, 4.5×10⁻⁶, 6.5×10⁻⁷ M, respectively. Compared to the Abcam AChE and the commercialized kit of Sichen with same activity units showing the IC₅₀ for insecticides mentioned above as 5.1×10⁻⁷, 8.2×10⁻⁷, 5.4×10⁻⁷, 6.5×10⁻⁶ M, respectively, and 2.3×10⁻⁶, 9.5×10⁻⁷, 2.8×10⁻⁶, 5.9×10⁻⁶ M, respectively. These results warrantee the present analysis platform as an easy, rapid, and practical test for the insecticide residues in vegetables and fruits with an affordable cost.

The results also showed a strategy that without the TM and CTD of GP64, a foreign membrane protein could still be possible to display on insect cell surface. However, the replacement of TM and CTD of a foreign protein with that of GP64 may stabilize the displayed proteins so they can sustain to more physical or chemical treatments such as the washing or the drug-adding step. Nevertheless, either way save a big effort for membrane protein purification, which is a tedious and expensive process. Frequently with a big loss of the target protein. This platform has several advantages over the previous systems. First, the AChE applied here is a much more sensitive mutant against that acquired from fly brains; second, insect cells that have displayed AChE is attached to the bottoms of 96-well microplates, therefore, the immobilization processes typically required for a biosensor is not necessary, tedious purification process is also not necessary; third, the AChE, which is displayed on insect cells could be freeze dried for convenient shipping; fourth, equipment, such as spectrophotometer is not needed; fifth, the cost is probably the very lowest among all known systems. In conclusion, we have developed a sensitive insect cell surface-display AChE platform that is promising for the future rapid determination of OP and CB insecticide residues, and suggested a practical strategy for improving the performance of the surface-display membrane proteins.

It will be apparent to those skilled in the art that various modifications and variations can be made to the disclosed embodiments. It is intended that the specification and examples be considered as exemplary only, with a true scope of the disclosure being indicated by the following claims and their equivalents.

### REFERENCES

The references listed below and referred to herein are hereby incorporated into this specification by reference unless this specification expressly provides otherwise.
1. Shani, A. Integrated pest management using pheromones. Chemtech 28, 30-35 (1998).
2. Donaldson, D., Kiely, T. & Grube, A. Pesticides Industry Sales and Usage: 1998 and 1999 Market Estimates. (ed. Agency, U.S.E.P.) (Washington, D C, 2002).
3. Fournier, D. & Mutero, A. Modification of acetylcholinesterase as a mechanism of resistance to insecticides. Comparative Biochemistry and Physiology Part C: Pharmacology, Toxicology and Endocrinology 108, 19-31 (1994).
4. Jiang, H., Liu, S., Zhao, P. & Pope, C. Recombinant expression and biochemical characterization of the catalytic domain of acetylcholinesterase-1 from the African malaria mosquito, Anopheles gambiae. Insect Biochemistry and Molecular Biology 39, 646-653 (2009).
5. Ellman, G. L. Tissue sulfhydryl groups. Archives of Biochemistry and Biophysics 82, 70-77 (1959).
6. Steele, R. W. & Smallman, B. N. Acetylcholinesterase from the house-fly head. Molecular properties of soluble forms. Biochimica et biophysica acta 445, 131-146 (1976).
7. Kost, T. A. & Condreay, J. P. Recombinant baculoviruses as mammalian cell gene-delivery vectors. Trends in Biotechnology 20, 173-180 (2002).
8. Possee, R. D., Thomas, C. J. & King, L. A. The use of baculovirus vectors for the production of membrane proteins in insect cells. Biochemical Society Transactions 27, 928 (1999).
9. Ayres, M. D., Howard, S. C., Kuzio, J., Lopez-Ferber, M. & Possee, R. D. The complete DNA sequence of Autographa californica nuclear polyhedrosis virus. Virology 202, 586-605 (1994).
10. Hall, L. M. & Spierer, P. The Ace locus of Drosophila melanogaster: structural gene for acetylcholinesterase with an unusual 5′ leader. The EMBO journal 5, 2949-2954 (1986).
11. Villatte, F., Marcel, V., Estrada-Mondaca, S. & Fournier, D. Engineering sensitive acetylcholinesterase for detection of organophosphate and carbamate insecticides. Biosensors and Bioelectronics 13, 157-164 (1998).
12. Chang, Y. J., Liu, C. Y., Chiang, B. L., Chao, Y. C. & Chen, C. C. Induction of IL-8 release in lung cells via activator protein-1 by recombinant baculovirus displaying severe acute respiratory syndrome-coronavirus spike proteins: identification of two functional regions. J Immunol 173, 7602-7614 (2004).
13. Grabherr, R., Ernst, W., Doblhoff-Dier, O., Sara, M. & Katinger, H. Expression of foreign proteins on the surface of Autographa californica nuclear polyhedrosis virus. Biotechniques 22, 730-735 (1997).
14. Yang, D. G., et al. Avian influenza virus hemagglutinin display on baculovirus envelope: cytoplasmic domain affects virus properties and vaccine potential. Mol Ther 15, 989-996 (2007).
15. Brestkin, A. P., Viniar, T. N. & Rozengart, E. V. [Interaction between various cholinesterases and reversible inhibitors of polymethylene-bis-(trimethylammonium) diiodide series]. Ukrainskii biokhimicheskii Zhurnal/55, 77-79 (1983).
16. Miao, Y., He, N. & Zhu, J. J. History and new developments of assays for cholinesterase activity and inhibition. Chemical reviews 110, 5216-5234 (2010).
17. Uete, T., Miyamato, Y., Onishi, M. & Shimano, N. Spectrophotometric micromethod for measuring cholinesterase activity in serum or plasma. Clinical chemistry 18, 454-458 (1972).
18. McOsker, D. E. & Daniel, L. J. A colorimetric micro method for the determination of cholinesterase. Archives of Biochemistry and Biophysics 79, 1-7 (1959).
19. Abernethy, M. H., George, P. M., Herron, J. L. & Evans, R. T. Plasma cholinesterase phenotyping with use of visible-region spectrophotometry. Clinical chemistry 32, 194-197 (1986).
20. Pavlov, V., Xiao, Y. & Willner, I. Inhibition of the acetycholine esterase-stimulated growth of Au nanoparticles: nanotechnology-based sensing of nerve gases. Nano letters 5, 649-653 (2005).
21. Guilbault, G. G. & Kramer, D. N. RESORUFIN BUTYRATE AND INDOXYL ACETATE AS FLUOROGENIC SUBSTRATES FOR CHOLINESTERASE. Analytical chemistry 37, 120-123 (1965).
22. Parvari, R., Pecht, I. & Soreq, H. A microfluorometric assay for cholinesterases, suitable for multiple kinetic determinations of picomoles of released thiocholine. Analytical biochemistry 133, 450-456 (1983).
23. Gill, R., Bahshi, L., Freeman, R. & Willner, I. Optical detection of glucose and acetylcholine esterase inhibitors by H2O2-sensitive CdSe/ZnS quantum dots. Angewandte Chemie (International ed. in English) 47, 1676-1679 (2008).

1. A method for pesticide detection in vitro, comprising:
(a) contacting a sample with cells expressing acetylcholinesterase;
(b) contacting an acetylcholinesterase substrate with the cells; and
(c) detecting a reaction product resulted from the acetylcholinesterase substrate.

2. The method of claim 1, wherein the acetylcholinesterase substrate comprises acetylcholine, acetylthiocholine (ATCh), propionylthiocholine or acetyl-3-methylcholine.

3. The method of claim 2, further comprising adding a fluorometric indicator before step c) so as to form the reaction product.

4. The method of claim 3, wherein the fluorometric indicator comprises 5,5′-dithio-bis-[2-nitrobenzoic acid] (DTNB), dithiodinicotnic acid (DTNA), 2,2′-dithiodipyridine (2-PDS), hydroxylamine, choline oxidase coupled with the peroxidase/phenol/aminoantipyrine, Au—NP seeds in the presence of AuCl4, resorufin butyrate, indoxyl acetate, N-[4-(7-diethylamino-4-methylcoumarin-3-yl) phenyl] maleimide, 10-acetyl-3,7-dihydroxyphenoxazine (Amplex Red reagent), quantum dots (QDs), thiol green indicator or AbRed indicator.

5. The method of claim 4, wherein the reaction product is 2-nitro-5-thiobenzoic acid (TNB) provided that the fluorometric indicator is DTNB.

6. The method of claim 1, further comprising quantifying the pesticide by referencing to a standard curve of an inhibition of acetylcholinesterase activity versus a concentration of the pesticide.

7. The method of claim 1, wherein the acetylcholinesterase is
(i) an acetylcholinesterase in full length,
(ii) an acetylcholinesterase with deletion of a glycosylphosphatidylinositol (GPI) anchor site; or
(iii) an acetylcholinesterase with deletion of a GPI anchor site and replaced with a transmembrane domain (TM) and a cytoplasmic domain (CTD) of a baculovirus GP64 protein.

8. The method of claim 7, wherein the acetylcholinesterase comprises amino acids of SEQ ID NO: 1, SEQ ID NO: 3, SEQ ID NO: 7, SEQ ID NO: 8, SEQ ID NO: 9, SEQ ID NO: 10, SEQ ID NO: 11, or SEQ ID NO: 12.

9.-10. (canceled)

11. The method of claim 1, wherein the pesticide comprises compounds that phosphorylates or carbamoylate acetylcholinesterase.

12. The method of claim 11, wherein the pesticide comprises organophosphates (OP) or carbamates (CB).

13. (canceled)

14. The method of claim 1, wherein two or more acetylcholinesterases are independently displayed on cells in different wells.

15. The method of claim 14, further comprising identifying the pesticide based on the reaction product of the two or more acetylcholinesterases.

16. The method of claim 14, wherein the two or more acetylcholinesterases are different acetylcholinesterase mutants or acetylcholinesterases derived from different organisms.

17. The method of claim 14, further comprising recording a coloring pattern of different pesticides as a fingerprint library.

18. The method of claim 17, further comprising identifying a concentration and species of an unknown pesticide via comparing to the fingerprint library.

19. The method of claim 17, further comprising identifying a concentration and species of the pesticides with different coloring patterns by machine learning.

20. The method of claim 19, wherein the machine learning is conducted by a software comprising Logistic Regression, Decision Tree, Support Vector Machine, Random or eXtreme Gradient Boosting.

21. The method of claim 1, wherein the acetylcholinesterases are independently displayed in various activities on cells in different wells.

22. The method of claim 21, wherein different cell concentrations were used for expression of acetylcholinesterases with various activities.

23.-26. (canceled)

27. A kit for pesticide detection, comprising:
an acetylcholinesterase expressed on cells, and
an acetylcholinesterase substrate.

28. The kit of claim 27, further comprising a fluorometric indicator.

29. The kit of claim 27, wherein the pesticide comprises organophosphates (OP) or carbamates (CB).

30. (canceled)


SEQUENCE LISTING (18 sequences)

SEQ ID NO: 1 - protein, 638 aa, Artificial Sequence
  AchE-FL amino acid sequence
MKFLVNVALVFMVVYISYIYAHHHHHHVIDRLVVQTSSGPVRGRSVTVQGREVHVYTGIPYAKPPVEDLRFRKPVPAEPWHGVLDATGLSATCVQERYEYFPGFSGEEIWNPNTNVSEDCLYINVWAPAKARLRHGRGANGGEHPNGKQADTDHLIHNGNPQNTTNGLPILIWIYGGGFMTGSATLDIYNADIMAAVGNVIVASFQYRVGAFGFLHLAPEMPSEFAEEAPGNVGLWDQALAIRWLKDNAHAFGGNPEWMTLFGESAGSSSVNAQLMSPVTRGLVKRGMMQSGTMNAPWSHMTSEKAVEIGKALINDCNCNASMLKTNPAHVMSCMRSVDAKTISVQQWNSYSGILSFPSAPTIDGAFLPADPMTLMKTADLKDYDILMGNVRDEGTFFLLYDFIDYFDKDDATALPRDKYLEIMNNIFGKATQAEREAIIFQYTSWEGNPGYQNQQQIGRAVGDHFFTCPTNEYAQALAERGASVHYYYFTHRTSTSLWGEWMGVLHGDEIEYFFGQPLNNSLQYRPVERELGKRMLSAVIEFAKTGNPAQDGEEWPNFSKEDPVYYIFSTDDKIEKLARGPLAARCSFWNDYLPKVRSWAGTCDGDSGSASISPRLQLLGIAALIYICAALRTKRVF

SEQ ID NO: 2 - DNA, 1917 bp, Artificial Sequence
  AchE-FL nucleic acid sequence
atgaaattcttagtcaacgttgcccttgtttttatggtcgtatacatttcttacatctatgcgcatcaccatcaccatcacgtcatcgatcgcctggttgtccagaccagctctggacctgttcgtggccgctcagtgacagtccaaggtcgtgaagttcacgtttacactggtatcccttacgctaagccaccagttgaagatttgaggttcagaaagcctgttccagctgaaccatggcacggtgttttagatgccacaggattaagcgccacatgcgtgcaagaaagatacgaatacttccccggtttttcaggtgaagaaatttggaaccctaacactaatgttagtgaagactgtctgtacattaacgtgtgggctcctgctaaggctagattgaggcacggacgtggtgctaacggtggcgaacaccctaacggaaaacaggctgatactgaccacctgatccacaacggaaaccctcaaaacactactaacggcctgccaatcctcatttggatctacggcggtggattcatgactggctcagctacattggacatctataacgctgacatcatggccgccgttggtaacgtgatcgttgcttcattccagtaccgcgtcggtgctttcggattcctccacttggctcctgaaatgccttctgaatttgctgaagaagctcctggcaacgtcggtctctgggaccaggctctggccatccgttggttgaaagacaacgctcacgctttcggcggcaaccctgaatggatgacattgttcggtgaatctgctggctccagcagcgtgaacgcccagctgatgagcccagtgacccgcggtctggttaagcgcggcatgatgcagtctggcacaatgaacgccccatggagccacatgactagcgagaaggccgtggaaatcggaaaggccctgatcaacgactgtaactgtaacgctagcatgctgaagactaaccctgcccacgtgatgagctgcatgcgctctgttgacgctaagaccatctccgtgcagcaatggaactcctacagcggaatcctctccttcccttctgctccaaccatcgacggtgccttcctccccgctgaccctatgacactcatgaagaccgctgacctgaaggactacgacatcctcatgggcaacgtgcgtgacgaaggtaccttcttcctgctctacgacttcatcgactacttcgacaaggacgacgccacagccctcccccgtgacaagtacctggaaatcatgaacaacatcttcggcaaggctactcaggccgagcgtgaggccatcatcttccagtacacatcctgggagggtaacccaggttaccaaaaccagcaacagatcggtcgtgctgttggtgaccacttcttcacatgccccacaaacgagtacgctcaggccctggccgaacgcggcgcctctgtccactactactacttcacccaccgcacctccacatctctgtggggtgagtggatgggtgtcctgcacggcgatgagatcgaatacttcttcggccagcccctcaacaatagcttacagtaccgccccgttgagcgtgaactaggtaaaaggatgctgagcgccgtgattgagttcgctaagacaggtaacccagcccaggatggtgaagaatggcctaactttagtaaggaagaccccgtttattacatctttagcactgatgataagattgaaaaattagctagaggacccctagccgctaggtgctcattctggaatgactacctccctaaggtgcgtagttgggctggcacatgtgacggagatagcggctccgcttctatctcacctagactgcagctgctcggaatcgctgccctgatctacatctgcgccgctctgagaactaagcgcgtgttctaa

SEQ ID NO: 3 - protein, 637 aa, Artificial Sequence
  AchE-6MC amino acid sequence
MKFLVNVALVFMVVYISYIYAHHHHHHVIDRLVVQTSSGPVRGRSVTVQGREVHVYTGIPYAKPPVEDLRFRKPVPAEPWHGVLDATGLSATCVQERYEYFPGFSGEEIWNPNTNVSEDCLYINVWAPAKARLRHGRGANGGEHPNGKQADTDHLIHNGNPQNTTNGLPILIWIYGGGFMTGSATLDIYNADIMAAVGNVIVASFQYRVGAFGFLHLAPEMPSEFAEEAPGNVGLWDQALAIRWLKDNAHAFGGNPEWMTLFGESAGSSSVNAQLMSPVTRGLVKRGMMQSGTMNAPWSHMTSEKAVEIGKALINDCNCNASMLKTNPAHVMSCMRSVDAKTISVQQWNSYSGILSFPSAPTIDGAFLPADPMTLMKTADLKDYDILMGNVRDEGTFFLLYDFIDYFDKDDATALPRDKYLEIMNNIFGKATQAEREAIIFQYTSWEGNPGYQNQQQIGRAVGDHFFTCPTNEYAQALAERGASVHYYYFTHRTSTSLWGEWMGVLHGDEIEYFFGQPLNNSLQYRPVERELGKRMLSAVIEFAKTGNPAQDGEEWPNFSKEDPVYYIFSTDDKIEKLARGPLAARCSFWNDYLPKVRSWAGTCDGDFMFGHVVNFVIILIVILFLYCMIRNRNRQY

SEQ ID NO: 4 - DNA, 1914 bp, Artificial Sequence
  AchE-6MC nucleic acid sequence
atgaaattcttagtcaacgttgcccttgtttttatggtcgtatacatttcttacatctatgcgcatcaccatcaccatcacgtcatcgatcgcctggttgtccagaccagctctggacctgttcgtggccgctcagtgacagtccaaggtcgtgaagttcacgtttacactggtatcccttacgctaagccaccagttgaagatttgaggttcagaaagcctgttccagctgaaccatggcacggtgttttagatgccacaggattaagcgccacatgcgtgcaagaaagatacgaatacttccccggtttttcaggtgaagaaatttggaaccctaacactaatgttagtgaagactgtctgtacattaacgtgtgggctcctgctaaggctagattgaggcacggacgtggtgctaacggtggcgaacaccctaacggaaaacaggctgatactgaccacctgatccacaacggaaaccctcaaaacactactaacggcctgccaatcctcatttggatctacggcggtggattcatgactggctcagctacattggacatctataacgctgacatcatggccgccgttggtaacgtgatcgttgcttcattccagtaccgcgtcggtgctttcggattcctccacttggctcctgaaatgccttctgaatttgctgaagaagctcctggcaacgtcggtctctgggaccaggctctggccatccgttggttgaaagacaacgctcacgctttcggcggcaaccctgaatggatgacattgttcggtgaatctgctggctccagcagcgtgaacgcccagctgatgagcccagtgacccgcggtctggttaagcgcggcatgatgcagtctggcacaatgaacgccccatggagccacatgactagcgagaaggccgtggaaatcggaaaggccctgatcaacgactgtaactgtaacgctagcatgctgaagactaaccctgcccacgtgatgagctgcatgcgctctgttgacgctaagaccatctccgtgcagcaatggaactcctacagcggaatcctctccttcccttctgctccaaccatcgacggtgccttcctccccgctgaccctatgacactcatgaagaccgctgacctgaaggactacgacatcctcatgggcaacgtgcgtgacgaaggtaccttcttcctgctctacgacttcatcgactacttcgacaaggacgacgccacagccctcccccgtgacaagtacctggaaatcatgaacaacatcttcggcaaggctactcaggccgagcgtgaggccatcatcttccagtacacatcctgggagggtaacccaggttaccaaaaccagcaacagatcggtcgtgctgttggtgaccacttcttcacatgccccacaaacgagtacgctcaggccctggccgaacgcggcgcctctgtccactactactacttcacccaccgcacctccacatctctgtggggtgagtggatgggtgtcctgcacggcgatgagatcgaatacttcttcggccagcccctcaacaatagcttacagtaccgccccgttgagcgtgaactaggtaaaaggatgctgagcgccgtgattgagttcgctaagacaggtaacccagcccaggatggtgaagaatggcctaactttagtaaggaagaccccgtttattacatctttagcactgatgataagattgaaaaattagctagaggacccctagccgctaggtgctcattctggaatgactacctccctaaggtgcgtagttgggctggcacatgtgacggagatttcatgtttggtcatgtagttaactttgtaattatattaattgtgattttatttttgtactgtatgattagaaaccgtaatagacaatattaa

SEQ ID NO: 5 - DNA, 9475 bp, Artificial Sequence
  pAchE-FL whole nucleic acid sequence
aacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatattcacttgtacagctcgtccatgccgccggtggagtggcggcctattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcggatctgacgggaattcctgcagcccgggggatccggtaccggtatgaacacgactcgaatcagactgatggtgttcgtcaccagtctcggctttttatagtgcggatggcccccactctgttgcgataagaaccgagcgacgccattggtcgatgagtacctcggcgcgttcgtactgttccacgatggtgtagtcctcgttgtgggaggtgatgtccaacttgatgttgacgttgtaggcgccgggcagccatcctgttttacaagtagaattctacccgtaaagcgagtttagttttgaaaaacaaatgacatcatttgtataatgacatcatcccctgattgtgttttacaagtagaattctatccgtaaagcgagttcagttttgaaaacaaatgagtcttacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgagccgtgtgcaaaacatgacatcagcttatgagtcataattaatcgtgcgctacaagtagaattctactcgtaaagcgagttgaaggatcatatttagttgcgtttatgagataagatctagagctcgactagaatcccaaaacaaactggttattgtggtaggtcatttgtttggcagaaagaaaactcgagaaatttctctggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatgccaccatgaaattcttagtcaacgttgcccttgtttttatggtcgtatacatttcttacatctatgcgcatcaccatcaccatcacgtcatcgatcgcctggttgtccagaccagctctggacctgttcgtggccgctcagtgacagtccaaggtcgtgaagttcacgtttacactggtatcccttacgctaagccaccagttgaagatttgaggttcagaaagcctgttccagctgaaccatggcacggtgttttagatgccacaggattaagcgccacatgcgtgcaagaaagatacgaatacttccccggtttttcaggtgaagaaatttggaaccctaacactaatgttagtgaagactgtctgtacattaacgtgtgggctcctgctaaggctagattgaggcacggacgtggtgctaacggtggcgaacaccctaacggaaaacaggctgatactgaccacctgatccacaacggaaaccctcaaaacactactaacggcctgccaatcctcatttggatctacggcggtggattcatgactggctcagctacattggacatctataacgctgacatcatggccgccgttggtaacgtgatcgttgcttcattccagtaccgcgtcggtgctttcggattcctccacttggctcctgaaatgccttctgaatttgctgaagaagctcctggcaacgtcggtctctgggaccaggctctggccatccgttggttgaaagacaacgctcacgctttcggcggcaaccctgaatggatgacattgttcggtgaatctgctggctccagcagcgtgaacgcccagctgatgagcccagtgacccgcggtctggttaagcgcggcatgatgcagtctggcacaatgaacgccccatggagccacatgactagcgagaaggccgtggaaatcggaaaggccctgatcaacgactgtaactgtaacgctagcatgctgaagactaaccctgcccacgtgatgagctgcatgcgctctgttgacgctaagaccatctccgtgcagcaatggaactcctacagcggaatcctctccttcccttctgctccaaccatcgacggtgccttcctccccgctgaccctatgacactcatgaagaccgctgacctgaaggactacgacatcctcatgggcaacgtgcgtgacgaaggtaccttcttcctgctctacgacttcatcgactacttcgacaaggacgacgccacagccctcccccgtgacaagtacctggaaatcatgaacaacatcttcggcaaggctactcaggccgagcgtgaggccatcatcttccagtacacatcctgggagggtaacccaggttaccaaaaccagcaacagatcggtcgtgctgttggtgaccacttcttcacatgccccacaaacgagtacgctcaggccctggccgaacgcggcgcctctgtccactactactacttcacccaccgcacctccacatctctgtggggtgagtggatgggtgtcctgcacggcgatgagatcgaatacttcttcggccagcccctcaacaatagcttacagtaccgccccgttgagcgtgaactaggtaaaaggatgctgagcgccgtgattgagttcgctaagacaggtaacccagcccaggatggtgaagaatggcctaactttagtaaggaagaccccgtttattacatctttagcactgatgataagattgaaaaattagctagaggacccctagccgctaggtgctcattctggaatgactacctccctaaggtgcgtagttgggctggcacatgtgacggagatagcggctccgcttctatctcacctagactgcagctgctcggaatcgctgccctgatctacatctgcgccgctctgagaactaagcgcgtgttctaattaattgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 6 - DNA, 9472 bp, Artificial Sequence
  pAchE-6MC whole nucleic acid sequence
aacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaatgagacgcacaaactaatatcacaaactggaaatgtctatcaatatatagttgctgatattcacttgtacagctcgtccatgccgccggtggagtggcggcctattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtagcgctagcggatctgacgggaattcctgcagcccgggggatccggtaccggtatgaacacgactcgaatcagactgatggtgttcgtcaccagtctcggctttttatagtgcggatggcccccactctgttgcgataagaaccgagcgacgccattggtcgatgagtacctcggcgcgttcgtactgttccacgatggtgtagtcctcgttgtgggaggtgatgtccaacttgatgttgacgttgtaggcgccgggcagccatcctgttttacaagtagaattctacccgtaaagcgagtttagttttgaaaaacaaatgacatcatttgtataatgacatcatcccctgattgtgttttacaagtagaattctatccgtaaagcgagttcagttttgaaaacaaatgagtcttacctaaacacgttaataatcttctgatatcagcttatgactcaagttatgagccgtgtgcaaaacatgagataagtttatgacatcatccactgatcgtgcgttacaagtagaattctactcgtaaagccagttcggttatgagccgtgtgcaaaacatgacatcagcttatgactcatacttgattgtgttttacgcgtagaattctactcgtaaagcgagttcggttatgagccgtgtgcaaaacatgacatcagcttatgagtcataattaatcgtgcgctacaagtagaattctactcgtaaagcgagttgaaggatcatatttagttgcgtttatgagataagatctagagctcgactagaatcccaaaacaaactggttattgtggtaggtcatttgtttggcagaaagaaaactcgagaaatttctctggccgttattcgttattctctcttttctttttgggtctctccctctctgcactaatgctctctcactctgtcacacagtaaacggcatactgctctcgttggttcgagagagcgcgcctcgaatgttcgcgaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatgccaccatgaaattcttagtcaacgttgcccttgtttttatggtcgtatacatttcttacatctatgcgcatcaccatcaccatcacgtcatcgatcgcctggttgtccagaccagctctggacctgttcgtggccgctcagtgacagtccaaggtcgtgaagttcacgtttacactggtatcccttacgctaagccaccagttgaagatttgaggttcagaaagcctgttccagctgaaccatggcacggtgttttagatgccacaggattaagcgccacatgcgtgcaagaaagatacgaatacttccccggtttttcaggtgaagaaatttggaaccctaacactaatgttagtgaagactgtctgtacattaacgtgtgggctcctgctaaggctagattgaggcacggacgtggtgctaacggtggcgaacaccctaacggaaaacaggctgatactgaccacctgatccacaacggaaaccctcaaaacactactaacggcctgccaatcctcatttggatctacggcggtggattcatgactggctcagctacattggacatctataacgctgacatcatggccgccgttggtaacgtgatcgttgcttcattccagtaccgcgtcggtgctttcggattcctccacttggctcctgaaatgccttctgaatttgctgaagaagctcctggcaacgtcggtctctgggaccaggctctggccatccgttggttgaaagacaacgctcacgctttcggcggcaaccctgaatggatgacattgttcggtgaatctgctggctccagcagcgtgaacgcccagctgatgagcccagtgacccgcggtctggttaagcgcggcatgatgcagtctggcacaatgaacgccccatggagccacatgactagcgagaaggccgtggaaatcggaaaggccctgatcaacgactgtaactgtaacgctagcatgctgaagactaaccctgcccacgtgatgagctgcatgcgctctgttgacgctaagaccatctccgtgcagcaatggaactcctacagcggaatcctctccttcccttctgctccaaccatcgacggtgccttcctccccgctgaccctatgacactcatgaagaccgctgacctgaaggactacgacatcctcatgggcaacgtgcgtgacgaaggtaccttcttcctgctctacgacttcatcgactacttcgacaaggacgacgccacagccctcccccgtgacaagtacctggaaatcatgaacaacatcttcggcaaggctactcaggccgagcgtgaggccatcatcttccagtacacatcctgggagggtaacccaggttaccaaaaccagcaacagatcggtcgtgctgttggtgaccacttcttcacatgccccacaaacgagtacgctcaggccctggccgaacgcggcgcctctgtccactactactacttcacccaccgcacctccacatctctgtggggtgagtggatgggtgtcctgcacggcgatgagatcgaatacttcttcggccagcccctcaacaatagcttacagtaccgccccgttgagcgtgaactaggtaaaaggatgctgagcgccgtgattgagttcgctaagacaggtaacccagcccaggatggtgaagaatggcctaactttagtaaggaagaccccgtttattacatctttagcactgatgataagattgaaaaattagctagaggacccctagccgctaggtgctcattctggaatgactacctccctaaggtgcgtagttgggctggcacatgtgacggagatttcatgtttggtcatgtagttaactttgtaattatattaattgtgattttatttttgtactgtatgattagaaaccgtaatagacaatattaattaattgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 7 - protein, 583 aa, Homo sapiens
EGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 8 - protein, 583 aa, Rattus norvegicus
EGREDPQLLVRVRGGQLRGIRLKAPGGPVSAFLGIPFAEPPVGSRRFMPPEPKRPWSGILDATTFQNVCYQYVDTLYPGFEGTEMWNPNRELSEDCLYLNVWTPYPRPTSPTPVLIWIYGGGFYSGASSLDVYDGRFLAQVEGTVLVSMNYRVGTFGFLALPGSREAPGNVGLLDQRLALQWVQENIAAFGGDPMSVTLFGESAGAASVGMHILSLPSRSLFHRAVLQSGTPNGPWATVSAGEARRRATLLARLVGCPPGGAGGNDTELISCLRTRPAQDLVDHEWHVLPQESIFRFSFVPVVDGDFLSDTPDALINTGDFQDLQVLVGVVKDEGSYFLVYGVPGFSKDNESLISRAQFLAGVRIGVPQASDLAAEAVVLHYTDWLHPEDPAHLRDAMSAVVGDHNVVCPVAQLAGRLAAQGARVYAYIFEHRASTLTWPLWMGVPHGYEIEFIFGLPLDPSLNYTVEERIFAQRLMQYWTNFARTGDPNDPRDSKSPRWPPYTTAAQQYVSLNLKPLEVRRGLRAQTCAFWNRFLPKLLSATDTLDEAERQWKAEFHRWSSYMVHWKNQFDHYSKQERCSDL

SEQ ID NO: 9 - protein, 606 aa, Apis mellifera
NAVPSSQRGNVHNDPLVVETTSGLVRGFPRTVLDKEVHVFYGIPFAKPPIGPLRFRKPLPIEPWHGVLNATVLPNSCYQERYEYFPGFPGEEMWNPNTNISEDCLYLNIWVPQKYRLRHKGDGSPGGNGGPRNGLLPLLVWIYGGGFMSGTATLDVYNADIMAATSNVIIASMQYRVGAFGFLYLNKHFTNSEEAPGNMGLWDQALALRWLRDNAEAFGGDPELITIFGESAGGSSVSLHLISPVTRGLVRRGILQSGTLNAPWSYMSGEKANEVATILVDDCGCNSTMLNENPARVMACMRSVDAKTISVQQWNSYWGILGFPSAPTIDGIFLPKHPLDLLREADFKDTEILIGNNENEGTYFILYDFNDIFEKDQASFLERERFLGIINNIFKNMSQIEREAITFQYTDWEEVYNGYIYQKMVADVVGDYFFICPSIHFAQLFADRGMKVYYYFFTQRTSTNLWGEWMGVLHGDEVEYVFGHPLNKSLKYSDKERDLSLRMILYFSEFAYLGKPTKEDSEWPSYSRDEPKYFIFDAEKTGLGKGPRTTYCAFWNEFLPKLKGIPDPAPNTCKVIASSVSAGEEGLGNSLAITALSSILVLSRVI

SEQ ID NO: 10 - protein, 615 aa, Spodoptera frugiperda
RSWANHHDTTTSTTQTTPTTSQAPKNFHNDPLIVETKSGLVKGYAKTVMGREVHIFTGIPFAKPPLGPLRFRKPVPIDPWHGVLEATAMPNSCYQERYEYFPGFEGEEMWNPNTNISEDCLYLNIWVPQHLRVRHHQDKPLTERPKVPILVWIYGGGYMSGTATLDLYKADIMASSSDVIVASMQYRVGAFGFLYLNKYFSPGSEEAPGNMGLWDQQLAIRWIKDNARAFGGDPELITLFGESAGGGSVSLHMLSPEMKGLFKRGILQSGTLNAPWSWMTGERAQDIGKVLVDDCNCNSSLLAADPSLVMDCMRGVDAKTISVQQWNSYTGILGFPSAPTVDGVFLPKDPDTMMKEGHFHNTEVLLGSNQDEGTYFLLYDFLDYFEKDGPSFLQREKFLEIVDTIFKDFSKIKREAIVFQYTDWEEITDGYLNQKMIADVVGDYFFVCPTNYFAEVLADSGVEVYYYYFTHRTSTSLWGEWMGVMHGDEMEYVFGHPLNMSLQYHTRERDLAAHIMQSFTRFALTGKPHKPDEKWPLYSRSSPHYYTYTADGTSGPAGPRGPRASACAFWNDFLNKLNELEHMPCDGAVTGPYSSVAGTTLPIVLLTTLATTVAL

SEQ ID NO: 11 - protein, 688 aa, Daphnia magna
QQLASAYRLGQRPMDNVTVLVSDNRKDDGVSIMGPLVPYQETDQWKHEQQTVFQEGERIRRDESSSRNGGSSSGGNKGRPGSIDDTNIRLLVQTLNGRVRGTTKTALGETVDVFLGIPFAKPPLGNLRFKKPVPIDPWQGVYDAQSLPNSCQQERYDVFPGFRGEEMWNPNTPISEDCLYLNLWVPTKLRHANNNNSGNGTVLIWIYGGGYMSGTSTLEVYDALILAATNDIIVVSINYRVGVFGFLYLDHEDAPGNMGLYDQALAIKWIKDNIRSFGGDPNSLTLFGESAGAGSVSVHLLSPVSGHLARRAIMQSGSVNAPWSYMTAETSKKIAVALVNDVGCNSSMVGIDTPSVMECMRSVQAKNLSLIQWNSYWGILGFPSAPTIDGVFLPKHPKDMLKEGGFSDAEILVGTNQDEGTYFILYDFIQYFKKDDPSVLEREKFLEIINTIFKTWSQLEREAIIFQYTDWDHIENGYLNQKMIGDVVGDYYFTCPTNYFAQKFAEHGTNVYYYHFTQRSSTNPWGQWMGVMHADEVEYVFGHPLNMSRDYTASERELSRRVMKYFATFAKTGKPVADDTLWPIYSRNEPRYFILNGEVRGIGHGPRATACAFWNEFMPLITARQSTNQCDPDAMLSLITARQSTNQCDPDAMLSLRGSASSIAKSSGRRGIFPIVLCWLLMAVLKVI

SEQ ID NO: 12 - protein, 618 aa, Bactrocera dorsalis
VIDRLVVQTSSGPVRGRSVTVQGREVHVYTGIPYAKPPLDDLRFRKPVPAEPWHGVLDATRLPATCVQERYEYFPGFSGEEIWNPNTNVSEDCLYINVWAPAKARLRHGRGANGGEHSNKADTDHLIHNGNPQNTTNGLPVLIWIYGGGFMTGTATLDIYNADIMSAVGNVIVASFQYRVGAFGFLHLSPAMPGYEEEAPGNVGLWDQALAIRWLKTNAHAFGGNPEWMTLFGESAGSSSVNAQLVSPVTAGLVKRGMMQSGTMNAPWSHMTSEKAVEIGKALINDCNCNASLLSENPQAVMACMRAVDAKTISVQQWNSYSGILSFPSAPTIDGAFLPDHPMKMMETADLRGYDILMGNVRDEGTYFLLYDFIDYFDKDEATSLPRDKYLEIMNNIFGKVTQAEREAIIFRHTSWVGNPGLENQQQIGRAVGDHFFTCPTNEYAQALAERGASVHYYYFTHRTSTSLWGEWMGVLHGDEIEYFFGQPLNTSLQYRQVERELGKRMLNAVIEFAKTGNPATDGEEWPNFTKKDPVYYVFSTDDKEEKLQRGPLEGRCAFWNEYLREVRKWGSQCELKPSSASSLQQKQQHLLLQQRSIVTFMLTLSLVLGIPSVNAFF

SEQ ID NO: 13 - DNA, 33 bp, Artificial Sequence
  HAChEifp primer
caccatcaccatcacgtcatcgatcgcctggtt

SEQ ID NO: 14 - DNA, 34 bp, Artificial Sequence
  AChE2rp primer
cggatcaattaattagaacacgcgcttagttctc

SEQ ID NO: 15 - DNA, 33 bp, Artificial Sequence
  AChEdeGPIrp primer
atgaccaaacatgaaatctccgtcacatgtgcc

SEQ ID NO: 16 - DNA, 48 bp, Artificial Sequence
  pABhhp10HM6H2fp primer
actaagcgcgtgttctaattaattgatccgggttattagtacatttat

SEQ ID NO: 17 - DNA, 34 bp, Artificial Sequence
  HAChEvrp primer
caggcgatcgatgacgtgatggtgatggtgatgc

SEQ ID NO: 18 - DNA, 44 bp, Artificial Sequence
  pABhhp10HM6Hfp primer
acatgtgacggagatttcatgtttggtcatgtagttaactttgt